(19) United States
(12) Patent Application Publication
(10) Pub. No.: US 2016/0244774 A1
(43) Pub. Date: Aug. 25, 2016

(54) VECTOR COMPRISING SPECIFIC PROMOTER AND GENE ENCODING SPECIFIC PROTEIN, TRANSGENIC PLANT INTO WHICH THE VECTOR HAS BEEN INTRODUCED, AND METHOD FOR IMPROVING POLYISOPRENOID PRODUCTION BY INTRODUCING THE VECTOR INTO PLANT

(71) Applicant: SUMITOMO RUBBER INDUSTRIES, LTD., Kobe-shi (JP)

(72) Inventors: Yukino INOUE, Kobe-shi (JP); Akari OKADA, Kobe-shi (JP); Haruhiko YAMAGUCHI, Kobe-shi (JP); Satoshi KURODA, Kobe-shi (JP)

(73) Assignee: SUMITOMO RUBBER INDUSTRIES, LTD., Kobe-shi (JP)

(21) Appl. No.: 15/018,504

(22) Filed: Feb. 8, 2016

(30) Foreign Application Priority Data
Feb. 23, 2015 (JP) 2015-033074

(51) Int. Cl. C12N 15/82; C07K 14/415; C12N 9/04; C12N 9/90; C12N 15/52; C12N 9/10

(52) U.S. Cl. CPC C12N 15/8251; C12N 15/52; C12N 9/1085; C12N 9/0006; C12N 9/90; C07K 14/415; C12Y 205/01; C12Y 205/0101; C12Y 101/01034; C12Y 503/03002; C12Y 205/0102

(57) ABSTRACT

Provided is a vector capable of improving polyisoprenoid production through the introduction of the vector into a plant using gene recombination techniques. A vector comprising: a promoter of a gene encoding Hevein 2.1; and a gene encoding a protein involved in polyisoprenoid biosynthesis, the gene being functionally linked to the promoter.

### TECHNICAL FIELD

The present invention relates to a vector comprising a specific promoter and a gene encoding a specific protein, a transgenic plant into which the vector has been introduced, and a method for improving polyisoprenoid production in a plant by introducing the vector into the plant.

### BACKGROUND ART

Nowadays natural rubber (one example of polyisoprenoids) for use in industrial rubber products can be harvested from isoprenoid-producing plants, such as para rubber tree (Hevea brasiliensis) belonging to the family Euphorbiaceae, or Indian rubber tree (Ficus elastica) belonging to the family Moraceae.

At present, para rubber tree is practically the only one source of natural rubber for industrial rubber products. Para rubber tree is a plant that can grow only in limited areas such as in Southeast Asia and South America. Moreover, para rubber tree requires about seven years from planting to mature enough for rubber extraction, and the period during which natural rubber can be extracted is limited to 20 to 30 years. Although more natural rubber is expected to be needed mainly by developing countries in years to come, for the reason mentioned above it is difficult to greatly increase the production of natural rubber using para rubber tree.

Meanwhile, along with the recent development in gene engineering, it is now possible to transform natural plants by introducing desired exogenous genes into the natural plants.

Since natural rubber is obtained from latex produced from the laticifers or latex ducts of specific isoprenoid-producing plants such as para rubber tree and Indian rubber tree, the development of gene recombination techniques has been considered for improving latex productivity to improve natural rubber production, but there is still room for improvement.

For this reason, depletion of natural rubber sources is of concern and there is a need to develop techniques that enables an improvement in natural rubber production.

### SUMMARY OF INVENTION

### Technical Problem

An object of the present invention is to solve the above-described problem and provide a vector that can improve polyisoprenoid production through the introduction of the vector into a plant using gene recombination techniques. Another object is to provide a transgenic plant into which the vector has been introduced and to provide a method for improving polyisoprenoid production in a plant by introducing the vector into the plant.

### Solution to Problem

The inventors made various studies for improving natural rubber production and therefore focused on gene recombination techniques and conducted research and development to create a transgenic plant that is improved in natural rubber production by enhancing a part of the polyisoprenoid biosynthesis pathway to thereby improve polyisoprenoid production. As a result, they constructed a vector comprising a base sequence in which a gene encoding a protein involved in polyisoprenoid biosynthesis is linked so as to be controlled by a promoter of a gene encoding Hevein 2.1. Then, the inventors found that by introducing the constructed vector into a plant, the gene encoding a protein involved in polyisoprenoid biosynthesis in the vector can be expressed specifically in laticifers, thereby improving polyisoprenoid production in the plant.

The present invention relates to a vector, comprising: a promoter of a gene encoding Hevein 2.1; and a gene encoding a protein involved in polyisoprenoid biosynthesis, the gene being functionally linked to the promoter.

It is preferable that the promoter of the gene encoding Hevein 2.1 comprises any one of the following DNAs:

[A1] a DNA comprising the base sequence of base numbers 1 to 1680 represented by SEQ ID NO: 1;
[A2] a DNA hybridizing to a DNA comprising a base sequence complementary to the base sequence of base numbers 1 to 1680 represented by SEQ ID NO: 1 under stringent conditions, and having a promoter activity for laticifer-specific gene expression; and
[A3] a DNA comprising a base sequence having 80% or more sequence identity with the base sequence of base numbers 1 to 1680 represented by SEQ ID NO: 1, and having a promoter activity for laticifer-specific gene expression.

It is preferable that the gene encoding a protein involved in polyisoprenoid biosynthesis is at least one gene selected from the group consisting of a gene encoding farnesyl diphosphate synthase, a gene encoding geranylgeranyl diphosphate synthase, a gene encoding 3-hydroxy-3-methylglutaryl CoA reductase, a gene encoding isopentenyl diphosphate isomerase, a gene encoding cis-prenyltransferase, a gene encoding Small Rubber Particle Protein, and a gene encoding Rubber Elongation Factor.

It is preferable that the gene encoding farnesyl diphosphate synthase comprises anyone of the following DNAs:

[B1] a DNA comprising the base sequence of base numbers 1 to 1029 represented by SEQ ID NO: 2;
[B2] a DNA hybridizing to a DNA comprising a base sequence complementary to the base sequence of base numbers 1 to 1029 represented by SEQ ID NO: 2 under stringent conditions, and encoding a protein having an enzyme activity that catalyzes a reaction using isopentenyl diphosphate and dimethylallyl diphosphate as substrates or a reaction using isopentenyl diphosphate and geranyl diphosphate as substrates; and
[B3] a DNA comprising a base sequence having 80% or more sequence identity with the base sequence of base numbers 1 to 1029 represented by SEQ ID NO: 2, and encoding a protein having an enzyme activity that catalyzes a reaction using isopentenyl diphosphate and dimethylallyl diphosphate as substrates or a reaction using isopentenyl diphosphate and geranyl diphosphate as substrates.

It is preferable that the gene encoding geranylgeranyl diphosphate synthase comprises any one of the following DNAs:

[C1] a DNA comprising the base sequence of base numbers 1 to 1113 represented by SEQ ID NO: 4;
[C2] a DNA hybridizing to a DNA comprising a base sequence complementary to the base sequence of base numbers 1 to 1113 represented by SEQ ID NO: 4 under stringent conditions, and encoding a protein having an enzyme activity that catalyzes a reaction using isopentenyl diphosphate and dimethylallyl diphosphate as substrates, a reaction using isopentenyl diphosphate and geranyl diphosphate as substrates, or a reaction using isopentenyl diphosphate and farnesyl diphosphate as substrates; and
[C3] a DNA comprising a base sequence having 80% or more sequence identity with the base sequence of base numbers 1 to 1113 represented by SEQ ID NO: 4, and encoding a protein having an enzyme activity that catalyzes a reaction using isopentenyl diphosphate and dimethylallyl diphosphate as substrates, a reaction using isopentenyl diphosphate and geranyl diphosphate as substrates, or a reaction using isopentenyl diphosphate and farnesyl diphosphate as substrates.

It is preferable that the gene encoding 3-hydroxy-3-methylglutaryl CoA reductase comprises anyone of the following DNAs:

[D1] a DNA comprising the base sequence of base numbers 1 to 1728 represented by SEQ ID NO: 6;
[D2] a DNA hybridizing to a DNA comprising a base sequence complementary to the base sequence of base numbers 1 to 1728 represented by SEQ ID NO: 6 under stringent conditions, and encoding a protein having an enzyme activity that catalyzes a reaction of reduction of 3-hydroxy-3-methylglutaryl CoA;
[D3] a DNA comprising a base sequence having 80% or more sequence identity with the base sequence of base numbers 1 to 1728 represented by SEQ ID NO: 6, and encoding a protein having an enzyme activity that catalyzes a reaction of reduction of 3-hydroxy-3-methylglutaryl CoA;
[D4] a DNA comprising the base sequence of base numbers 1 to 1761 represented by SEQ ID NO: 7;
[D5] a DNA hybridizing to a DNA comprising a base sequence complementary to the base sequence of base numbers 1 to 1761 represented by SEQ ID NO: 7 under stringent conditions, and encoding a protein having an enzyme activity that catalyzes a reaction of reduction of 3-hydroxy-3-methylglutaryl CoA;
[D6] a DNA comprising a base sequence having 80% or more sequence identity with the base sequence of base numbers 1 to 1761 represented by SEQ ID NO: 7, and encoding a protein having an enzyme activity that catalyzes a reaction of reduction of 3-hydroxy-3-methylglutaryl CoA;
[D7] a DNA comprising the base sequence of base numbers 1 to 1821 represented by SEQ ID NO: 8;
[D8] a DNA hybridizing to a DNA comprising a base sequence complementary to the base sequence of base numbers 1 to 1821 represented by SEQ ID NO: 8 under stringent conditions, and encoding a protein having an enzyme activity that catalyzes a reaction of reduction of 3-hydroxy-3-methylglutaryl CoA;
[D9] a DNA comprising a base sequence having 80% or more sequence identity with the base sequence of base numbers 1 to 1821 represented by SEQ ID NO: 8, and encoding a protein having an enzyme activity that catalyzes a reaction of reduction of 3-hydroxy-3-methylglutaryl CoA;
[D10] a DNA comprising the base sequence of base numbers 1 to 1581 represented by SEQ ID NO: 9;
[D11] a DNA hybridizing to a DNA comprising a base sequence complementary to the base sequence of base numbers 1 to 1581 represented by SEQ ID NO: 9 under stringent conditions, and encoding a protein having an enzyme activity that catalyzes a reaction of reduction of 3-hydroxy-3-methylglutaryl CoA; and
[D12] a DNA comprising a base sequence having 80% or more sequence identity with the base sequence of base numbers 1 to 1581 represented by SEQ ID NO: 9, and encoding a protein having an enzyme activity that catalyzes a reaction of reduction of 3-hydroxy-3-methylglutaryl CoA.

It is preferable that the gene encoding isopentenyl diphosphate isomerase comprises anyone of the following DNAs:

[E1] a DNA comprising the base sequence of base numbers 1 to 705 represented by SEQ ID NO: 14;
[E2] a DNA hybridizing to a DNA comprising a base sequence complementary to the base sequence of base numbers 1 to 705 represented by SEQ ID NO: 14 under stringent conditions, and encoding a protein having an enzyme activity that catalyzes a reaction of isomerization of isopentenyl diphosphate or dimethylallyl diphosphate; and
[E3] a DNA comprising a base sequence having 80% or more sequence identity with the base sequence of base numbers 1 to 705 represented by SEQ ID NO: 14, and encoding a protein having an enzyme activity that catalyzes a reaction of isomerization of isopentenyl diphosphate or dimethylallyl diphosphate.

It is preferable that the gene encoding cis-prenyltransferase comprises any one of the following DNAs:

[F1] a DNA comprising the base sequence of base numbers 1 to 873 represented by SEQ ID NO: 16;
[F2] a DNA hybridizing to a DNA comprising a base sequence complementary to the base sequence of base numbers 1 to 873 represented by SEQ ID NO: 16 under stringent conditions, and encoding a protein having an enzyme activity that catalyzes a reaction of cis-chain elongation of isoprenoid compounds;
[F3] a DNA comprising a base sequence having 80% or more sequence identity with the base sequence of base numbers 1 to 873 represented by SEQ ID NO: 16, and encoding a protein having an enzyme activity that catalyzes a reaction of cis-chain elongation of isoprenoid compounds;
[F4] a DNA comprising the base sequence of base numbers 1 to 855 represented by SEQ ID NO: 66;
[F5] a DNA hybridizing to a DNA comprising a base sequence complementary to the base sequence of base numbers 1 to 855 represented by SEQ ID NO: 66 under stringent conditions, and encoding a protein having an enzyme activity that catalyzes a reaction of cis-chain elongation of isoprenoid compounds; and
[F6] a DNA comprising a base sequence having 80% or more sequence identity with the base sequence of base numbers 1 to 855 represented by SEQ ID NO: 66, and encoding a protein having an enzyme activity that catalyzes a reaction of cis-chain elongation of isoprenoid compounds.

It is preferable that the gene encoding Small Rubber Particle Protein comprises any one of the following DNAs:

[G1] a DNA comprising the base sequence of base numbers 1 to 615 represented by SEQ ID NO: 18;
[G2] a DNA hybridizing to a DNA comprising a base sequence complementary to the base sequence of base numbers 1 to 615 represented by SEQ ID NO: 18 under stringent conditions, and encoding a rubber particle-associated protein which is associated with rubber particles in latex; and
[G3] a DNA comprising a base sequence having 80% or more sequence identity with the base sequence of base numbers 1 to 615 represented by SEQ ID NO: 18, and encoding a rubber particle-associated protein which is associated with rubber particles in latex.

It is preferable that the gene encoding Rubber Elongation Factor comprises any one of the following DNAs:

[H1] a DNA comprising the base sequence of base numbers 1 to 417 represented by SEQ ID NO: 60;
[H2] a DNA hybridizing to a DNA comprising a base sequence complementary to the base sequence of base numbers 1 to 417 represented by SEQ ID NO: 60 under stringent conditions, and encoding a rubber particle-associated protein which is associated with rubber particles in latex; and
[H3] a DNA comprising a base sequence having 80% or more sequence identity with the base sequence of base numbers 1 to 417 represented by SEQ ID NO: 60, and encoding a rubber particle-associated protein which is associated with rubber particles in latex.

The present invention also relates to a transgenic plant into which any of the vectors described above has been introduced.

The present invention also relates to a method for improving polyisoprenoid production in a plant by introducing any of the vectors described above into the plant.

### Advantageous Effects of Invention

The vector of the present invention comprises a base sequence in which a gene encoding a protein involved in polyisoprenoid biosynthesis is functionally linked to a promoter of a gene encoding Hevein 2.1. Further, by introducing such a vector into a plant, the gene encoding a protein involved in polyisoprenoid biosynthesis in the vector can be expressed specifically in laticifers, thereby improving polyisoprenoid production in the plant.

### BRIEF DESCRIPTION OF DRAWINGS

FIG. 1 is a schematic diagram showing a part of the polyisoprenoid biosynthesis pathway;

FIG. 2 is a schematic diagram showing a part of the polyisoprenoid biosynthesis pathway;

FIG. 3 is a schematic diagram showing a part of the mevalonic acid pathway which is upstream of a polyisoprenoid synthesis pathway;

FIG. 4 is a schematic diagram showing a part of the polyisoprenoid biosynthesis pathway;

FIG. 5 is a schematic diagram showing a part of the polyisoprenoid biosynthesis pathway;

FIG. 6 is a schematic diagram showing a part of the polyisoprenoid biosynthesis pathway;

FIG. 7 is a schematic diagram showing a T-DNA region of an expression vector introduced into Agrobacterium used in Example 1;

FIG. 8 is a schematic diagram showing a T-DNA region of an expression vector introduced into Agrobacterium used in Example 2;

FIG. 9 is a schematic diagram showing a T-DNA region of an expression vector introduced into Agrobacterium used in Example 3, 8, 9, or 10;

FIG. 10 is a schematic diagram showing a T-DNA region of an expression vector introduced into Agrobacterium used in Example 4;

FIG. 11 is a schematic diagram showing a T-DNA region of an expression vector introduced into Agrobacterium used in Example 5 or 11;

FIG. 12 is a schematic diagram showing a T-DNA region of an expression vector introduced into Agrobacterium used in Example 6; and

FIG. 13 is a schematic diagram showing a T-DNA region of an expression vector introduced into Agrobacterium used in Example 7.

### DESCRIPTION OF EMBODIMENTS

### Vector

The vector of the present invention comprises a base sequence in which a gene encoding a protein involved in polyisoprenoid biosynthesis is functionally linked to a promoter of a gene encoding Hevein 2.1 (HEV2.1) having a promoter activity for laticifer-specific gene expression. By introducing such a vector into a plant for transformation, the gene encoding a protein involved in polyisoprenoid biosynthesis in the vector can be expressed specifically in laticifers, thereby improving polyisoprenoid production in the plant. This is presumably because if the expression of an exogenous gene introduced for improvement of latex productivity is promoted in sites other than laticifers, a certain load is imposed on the metabolism or the production of latex of the plant, thereby causing adverse effects.

In the present specification, the term “Hevein 2.1 (HEV2.1)” refers to a protein that is highly expressed in the laticifer cells of polyisoprenoid-producing plants such as para rubber tree (Hevea brasiliensis). The Hevein 2.1 is involved in aggregation of rubber particles and has antifungal activity.

In the present specification, the “promoter having a promoter activity for laticifer-specific gene expression” means that the promoter has an activity for controlling gene expression to cause the laticifer-specific expression of a desired gene when the desired gene is functionally linked to the promoter and introduced into a plant. The terms “laticifer-specific gene expression” means that the gene is expressed substantially exclusively in laticifers with no or little expression in sites other than laticifers in the plant. Also, “a gene is functionally linked to a promoter” means that the gene sequence is linked downstream of the promoter so that the gene is controlled by the promoter.

The vector of the present invention can be prepared by inserting the base sequence of a promoter of a gene encoding HEV2.1 and the base sequence of a gene encoding a protein involved in polyisoprenoid biosynthesis into a vector generally known as a plant transformation vector by conventional techniques. Examples of vectors usable for preparing the vector of the present invention include pBI vectors, binary vectors such as pGA482, pGAH, and pBIG, intermediate plasmids such as pLGV23Neo, pNCAT, and pMON200, pH35GS containing GATEWAY cassette, and the like.

The vector of the present invention may contain other base sequences as long as the vector comprises the base sequence of a promoter of a gene encoding HEV2.1 and the base sequence of a gene encoding a protein involved in polyisoprenoid biosynthesis. Usually, the vector contains sequences derived from the original vector in addition to these base sequences and further contains a restriction enzyme recognition sequence, a spacer sequence, a sequence of a marker gene, a sequence of a reporter gene, and so forth.

Examples of the marker gene include drug-resistant genes such as a kanamycin-resistant gene, hygromycin-resistant gene, and a bleomycin-resistant gene. The reporter gene is introduced to determine the expression site in a plant, and examples include a luciferase gene, a GUS (β-glucuronidase) gene, GFP (green fluorescent protein), RFP (red fluorescent protein), and so forth.

### (Promoter of Gene Encoding HEV2.1)

The promoter of the gene encoding HEV2.1 may preferably be derived from a plant, more preferably from a polyisoprenoid-producing plant, without particular limitation thereto. Among them, the promoter may further preferably be derived from para rubber tree, guayule, Russian dandelion, Canada goldenrod, common sowthistle, lettuce, or sunflower, particularly preferably from para rubber tree.

In the present specification, the polyisoprenoid-producing plant means a plant capable of producing a polyisoprenoid, and specific examples will be described later.

The promoter of the gene encoding HEV2.1 may preferably be any one of the following DNAs:

[A1] a DNA comprising the base sequence of base numbers 1 to 1680 represented by SEQ ID NO: 1;
[A2] a DNA hybridizing to a DNA comprising a base sequence complementary to the base sequence of base numbers 1 to 1680 represented by SEQ ID NO: 1 under stringent conditions, and having a promoter activity for laticifer-specific gene expression; and
[A3] a DNA comprising a base sequence having 80% or more sequence identity with the base sequence of base numbers 1 to 1680 represented by SEQ ID NO: 1, and having a promoter activity for laticifer-specific gene expression.

As used herein, the term “hybridizing” means a process in which a DNA hybridizes to a DNA having a specific base sequence or a part of the DNA. Accordingly, the DNA having a specific base sequence or part of the DNA may have a base sequence long enough to be usable as a probe in Northern or Southern blot analysis or as an oligonucleotide primer in polymerase chain reaction (PCR) analysis. The DNA used as a probe may have a length of at least 100 bases, preferably at least 200 bases, and more preferably at least 500 bases although it may be a DNA of at least 10 bases, and preferably of at least 15 bases in length.

Techniques to perform DNA hybridization experiments are well known. The hybridization conditions under which experiments are performed may be determined according to, for example, Molecular Cloning, 2nd ed. and 3rd ed. (2001), Methods for General and Molecular Bacteriology, ASM Press (1994), Immunology methods manual, Academic press (Molecular), and many other standard textbooks (all the above documents are incorporated herein by reference).

The stringent conditions may include, for example, an overnight incubation at 42° C. of a DNA-immobilized filter and a DNA probe in a solution containing 50% formamide, 5×SSC (750 mM sodium chloride, 75 mM sodium citrate), 50 mM sodium phosphate (pH 7.6), 5×Denhardt's solution, 10% dextran sulfate, and 20 μg/l denatured salmon sperm DNA, followed by washing the filter for example in a 0.2×SSC solution at approximately 65° C. Less stringent conditions may also be used. Changes in the stringency may be accomplished through the manipulation of formamide concentration (lower percentages of formamide result in lower stringency), salt concentrations or temperature. For example, low stringent conditions include an overnight incubation at 37° C. in a solution containing 6×SSCE (20×SSCE: 3 mol/l sodium chloride, 0.2 mol/l sodium dihydrogen phosphate, 0.02 mol/l EDTA, pH 7.4), 0.5% SDS, 30% formamide, and 100 μg/l denatured salmon sperm DNA, followed by washing in a 1×SSC solution containing 0.1% SDS at 50° C. In addition, to achieve even lower stringency, washes performed following hybridization may be done at higher salt concentrations (e.g. 5×SSC) in the above-mentioned low stringent conditions.

Variations in the above various conditions may be accomplished through the inclusion or substitution of blocking reagents used to suppress background in hybridization experiments. The inclusion of blocking reagents may require modification of the hybridization conditions for compatibility.

Like the DNA capable of hybridization under stringent conditions described above, it is known that some promoters with base sequences having a certain sequence identity with the original base sequence have similar promoter activity. In order to maintain the promoter activity, the sequence identity with the base sequence of base numbers 1 to 1680 represented by SEQ ID NO: 1 is at least 80% or more, preferably 90% or more, more preferably 95% or more, further preferably 98% or more, particularly preferably 99% or more.

The sequence identity between base sequences or amino acid sequences may be determined using the algorithm BLAST [Pro. Natl. Acad. Sci. USA, 90, 5873 (1993)] developed by Karlin and Altschul or FASTA [Methods Enzymol., 183, 63 (1990)] (all the above documents are incorporated herein by reference).

Whether a DNA hybridizing to the above-described DNA under stringent conditions or a DNA having 80% or more sequence identity with the above-described DNA has a promoter activity for laticifer-specific gene expression may be determined by conventional techniques, such as reporter assays using a reporter gene encoding β-galactosidase, luciferase, green fluorescent protein (GFP), or the like.

Conventional techniques may be employed to identify the base sequence of the promoter of the gene encoding HEV2.1, and, for example, a genomic DNA may be extracted from a growing plant by the CTAB (Cetyl Trimethyl Ammonium Bromide) method. Next, specific primers and random primers are designed based on the known base sequence of the gene encoding HEV2.1, and the gene including the HEV2.1 promoter is amplified by TAIL (Thermal Asymmetric Interlaced)-PCR using the extracted genomic DNA as a template to identify the base sequence.

### (Protein Involved in Polyisoprenoid Biosynthesis)

The gene encoding a protein involved in polyisoprenoid biosynthesis may preferably be at least one gene selected from the group consisting of a gene encoding farnesyl diphosphate synthase, a gene encoding geranylgeranyl diphosphate synthase, a gene encoding 3-hydroxy-3-methylglutaryl CoA reductase, a gene encoding isopentenyl diphosphate isomerase, a gene encoding cis-prenyltransferase, a gene encoding Small Rubber Particle Protein, and a gene encoding Rubber Elongation Factor. Among them, for further improved polyisoprenoid production, it may more preferably be at least one gene selected from the group consisting of a gene encoding 3-hydroxy-3-methylglutaryl CoA reductase, a gene encoding isopentenyl diphosphate isomerase, a gene encoding cis-prenyltransferase, and a gene encoding Rubber Elongation Factor, further preferably a gene encoding 3-hydroxy-3-methylglutaryl CoA reductase or a gene encoding cis-prenyltransferase, particularly preferably a gene encoding cis-prenyltransferase.

The inventors aimed at creation of a transgenic plant that is improved in natural rubber production by enhancing a part of the polyisoprenoid biosynthesis pathway to thereby improve polyisoprenoid production. First of all, the two pathways, mevalonic acid pathway (MVA pathway) and non-mevalonic acid pathway (MEP pathway) are known as pathways for biosynthesis of isoprenyl diphosphate (IPP), which is an important member of the polyisoprenoid biosynthesis pathway. The inventors focused on the MVA pathway and selected, from various proteins involved in the polyisoprenoid biosynthesis pathway, some proteins that are expected to have important roles to enhance the MVA pathway or a downstream part of the pathway.

Specifically, the following seven proteins were selected: farnesyl diphosphate synthase (FPS) and geranylgeranyl diphosphate synthase (GGPS), which are prenyltransferases involved in reactions in which IPP is linked to allylic substrates so that isoprene units are sequentially connected, and synthesize farnesyl diphosphate (FPP) and geranylgeranyl diphosphate (GGPP), respectively, which are considered as starting substrates for natural rubber; 3-hydroxy-3-methylglutaryl CoA reductase (HMGR) which is a rate-limiting factor in the MVA pathway; isopentenyl diphosphate isomerase (IPI) which is involved in isomerization of IPP and dimethylallyl diphosphate (DMAPP); cis-prenyltransferase (CPT) which is thought to be involved in chain elongation of isoprenoid compounds; Small Rubber Particle Protein (SRPP) and Rubber Elongation Factor (REF), which are known to be involved in polyisoprenoid biosynthesis. Then, vectors were constructed, each of which comprises a base sequence in which a gene encoding each of these proteins is linked so as to be under the control of a promoter of a gene encoding HEV 2.1. The constructed vectors could each be introduced into a plant to improve polyisoprenoid production in the plant.

As mentioned above, the reactions in which isopentenyl diphosphate (IPP) is sequentially linked to allylic substrates proceed in the polyisoprenoid biosynthesis pathway. The enzymes catalyzing the reactions are collectively referred to as prenyltransferase in the sense that isoprene units are sequentially connected. In the present specification, the term “prenyltransferase” means a collective term of enzymes that each catalyze a condensation reaction between IPP and an isoprenyl diphosphate (the number of isoprene units is n) (allylic substrate) to synthesize a new isoprenyl diphosphate (the number of isoprene units is n+1) in which one isoprene unit is added.

The prenyltransferases are a group of enzymes that link isoprene units to synthesize various isoprenyl diphosphates such as geranyl diphosphate (GPP: C10), farnesyl diphosphate (FPP: C15), geranylgeranyl diphosphate (GGPP: C20), geranylfarnesyl diphosphate (GFPP: C25), hexaprenyl diphosphate (HPP: C30), or the like serving as basic precursors of terpenoids, and are positioned in the mainstream of terpenoid biosynthesis. Farnesyl diphosphate synthase (FPS), geranylgeranyl diphosphate synthase (GGPS), and so forth are classified as prenyltransferases.

In the present specification, the term “farnesyl diphosphate synthase (FPS)” refers to an enzyme that catalyzes a farnesyl diphosphate (FPP) biosynthesis reaction using isopentenyl diphosphate (IPP), dimethylallyl diphosphate (DMAPP), and geranyl diphosphate (GPP) as substrates.

Also, in the present specification, the term “geranylgeranyl diphosphate synthase (GGPS)” refers to an enzyme that catalyzes a geranylgeranyl diphosphate (GGPP) biosynthesis reaction using isopentenyl diphosphate (IPP), dimethylallyl diphosphate (DMAPP), geranyl diphosphate (GPP), and farnesyl diphosphate (FPP) as substrates.

Also, in the present specification, the term “3-hydroxy-3-methylglutaryl CoA reductase (HMG-CoA reductase, HMGR)” is one of rate-limiting enzymes in the mevalonic acid pathway (MVA pathway) and includes both 3-hydroxy-3-methylglutaryl CoA reductase (NADPH) (EC 1.1.1.34) and 3-hydroxy-3-methylglutaryl CoA reductase (EC 1.1.1.88).

Also, in the present specification, the term “isopentenyl diphosphate isomerase (IPP isomerase, IPI)” refers to an enzyme that catalyzes an isomerization reaction between isopentenyl diphosphate (IPP) and its isomer, dimethylallyl diphosphate (DMAPP).

Also, in the present specification, the term “cis-prenyltransferase (cis-type prenyltransferase, CPT)” refers to an enzyme that catalyzes a reaction of cis-chain elongation of isoprenoid compounds. As used herein, the term “isoprenoid compound” means a compound containing an isoprene unit (C₅H₈). Also, the term “cis isoprenoid” refers to a compound including an isoprenoid compound in which isoprene units are cis-bonded and examples include cis-farnesyl diphosphate, undecaprenyl diphosphate, natural rubber, and the like.

Also, in the present specification, the term “Small Rubber Particle Protein (SRPP)” refers to a rubber particle-associated protein which is associated with rubber particles in the latex of a polyisoprenoid-producing plant such as para rubber tree (Hevea brasiliensis).

Also, in the present specification, the term “Rubber Elongation Factor (REF)” refers to a rubber particle-associated protein which is associated with rubber particles in the latex of a polyisoprenoid-producing plant such as para rubber tree (Hevea brasiliensis).

### (Gene)

The gene encoding farnesyl diphosphate synthase, gene encoding geranylgeranyl diphosphate synthase, gene encoding 3-hydroxy-3-methylglutaryl CoA reductase, gene encoding isopentenyl diphosphate isomerase, gene encoding cis-prenyltransferase, gene encoding Small Rubber Particle Protein, and gene encoding Rubber Elongation Factor may each preferably be derived from a plant, more preferably from a polyisoprenoid-producing plant, without particular limitation thereto. Among others, the genes may each further preferably be derived from para rubber tree, guayule, Russian dandelion, Canada goldenrod, common sowthistle, lettuce, or sunflower, particularly preferably from para rubber tree.

The gene encoding farnesyl diphosphate synthase may preferably comprise any one of the following DNAs:

[B1] a DNA comprising the base sequence of base numbers 1 to 1029 represented by SEQ ID NO: 2;
[B2] a DNA hybridizing to a DNA comprising a base sequence complementary to the base sequence of base numbers 1 to 1029 represented by SEQ ID NO: 2 under stringent conditions, and encoding a protein having an enzyme activity that catalyzes a reaction using isopentenyl diphosphate and dimethylallyl diphosphate as substrates or a reaction using isopentenyl diphosphate and geranyl diphosphate as substrates; and
[B3] a DNA comprising a base sequence having 80% or more sequence identity with the base sequence of base numbers 1 to 1029 represented by SEQ ID NO: 2, and encoding a protein having an enzyme activity that catalyzes a reaction using isopentenyl diphosphate and dimethylallyl diphosphate as substrates or a reaction using isopentenyl diphosphate and geranyl diphosphate as substrates.

The gene encoding geranylgeranyl diphosphate synthase may preferably comprise any one of the following DNAs:

[C1] a DNA comprising the base sequence of base numbers 1 to 1113 represented by SEQ ID NO: 4;
[C2] a DNA hybridizing to a DNA comprising a base sequence complementary to the base sequence of base numbers 1 to 1113 represented by SEQ ID NO: 4 under stringent conditions, and encoding a protein having an enzyme activity that catalyzes a reaction using isopentenyl diphosphate and dimethylallyl diphosphate as substrates, a reaction using isopentenyl diphosphate and geranyl diphosphate as substrates, or a reaction using isopentenyl diphosphate and farnesyl diphosphate as substrates; and
[C3] a DNA comprising a base sequence having 80% or more sequence identity with the base sequence of base numbers 1 to 1113 represented by SEQ ID NO: 4, and encoding a protein having an enzyme activity that catalyzes a reaction using isopentenyl diphosphate and dimethylallyl diphosphate as substrates, a reaction using isopentenyl diphosphate and geranyl diphosphate as substrates, or a reaction using isopentenyl diphosphate and farnesyl diphosphate as substrates.

The gene encoding 3-hydroxy-3-methylglutaryl CoA reductase may preferably comprise any one of the following DNAs:

[D1] a DNA comprising the base sequence of base numbers 1 to 1728 represented by SEQ ID NO: 6;
[D2] a DNA hybridizing to a DNA comprising a base sequence complementary to the base sequence of base numbers 1 to 1728 represented by SEQ ID NO: 6 under stringent conditions, and encoding a protein having an enzyme activity that catalyzes a reaction of reduction of 3-hydroxy-3-methylglutaryl CoA;
[D3] a DNA comprising a base sequence having 80% or more sequence identity with the base sequence of base numbers 1 to 1728 represented by SEQ ID NO: 6, and encoding a protein having an enzyme activity that catalyzes a reaction of reduction of 3-hydroxy-3-methylglutaryl CoA;
[D4] a DNA comprising the base sequence of base numbers 1 to 1761 represented by SEQ ID NO: 7;
[D5] a DNA hybridizing to a DNA comprising a base sequence complementary to the base sequence of base numbers 1 to 1761 represented by SEQ ID NO: 7 under stringent conditions, and encoding a protein having an enzyme activity that catalyzes a reaction of reduction of 3-hydroxy-3-methylglutaryl CoA;
[D6] a DNA comprising a base sequence having 80% or more sequence identity with the base sequence of base numbers 1 to 1761 represented by SEQ ID NO: 7, and encoding a protein having an enzyme activity that catalyzes a reaction of reduction of 3-hydroxy-3-methylglutaryl CoA;
[D7] a DNA comprising the base sequence of base numbers 1 to 1821 represented by SEQ ID NO: 8;
[D8] a DNA hybridizing to a DNA comprising a base sequence complementary to the base sequence of base numbers 1 to 1821 represented by SEQ ID NO: 8 under stringent conditions, and encoding a protein having an enzyme activity that catalyzes a reaction of reduction of 3-hydroxy-3-methylglutaryl CoA;
[D9] a DNA comprising a base sequence having 80% or more sequence identity with the base sequence of base numbers 1 to 1821 represented by SEQ ID NO: 8, and encoding a protein having an enzyme activity that catalyzes a reaction of reduction of 3-hydroxy-3-methylglutaryl CoA;
[D10] a DNA comprising the base sequence of base numbers 1 to 1581 represented by SEQ ID NO: 9;
[D11] a DNA hybridizing to a DNA comprising a base sequence complementary to the base sequence of base numbers 1 to 1581 represented by SEQ ID NO: 9 under stringent conditions, and encoding a protein having an enzyme activity that catalyzes a reaction of reduction of 3-hydroxy-3-methylglutaryl CoA; and
[D12] a DNA comprising a base sequence having 80% or more sequence identity with the base sequence of base numbers 1 to 1581 represented by SEQ ID NO: 9, and encoding a protein having an enzyme activity that catalyzes a reaction of reduction of 3-hydroxy-3-methylglutaryl CoA.

The gene encoding isopentenyl diphosphate isomerase may preferably comprise any one of the following DNAs:

[E1] a DNA comprising the base sequence of base numbers 1 to 705 represented by SEQ ID NO: 14;
[E2] a DNA hybridizing to a DNA comprising a base sequence complementary to the base sequence of base numbers 1 to 705 represented by SEQ ID NO: 14 under stringent conditions, and encoding a protein having an enzyme activity that catalyzes a reaction of isomerization of isopentenyl diphosphate or dimethylallyl diphosphate; and
[E3] a DNA comprising a base sequence having 80% or more sequence identity with the base sequence of base numbers 1 to 705 represented by SEQ ID NO: 14, and encoding a protein having an enzyme activity that catalyzes a reaction of isomerization of isopentenyl diphosphate or dimethylallyl diphosphate.

The gene encoding cis-prenyltransferase may preferably comprise any one of the following DNAs:

[F1] a DNA comprising the base sequence of base numbers 1 to 873 represented by SEQ ID NO: 16;
[F2] a DNA hybridizing to a DNA comprising a base sequence complementary to the base sequence of base numbers 1 to 873 represented by SEQ ID NO: 16 under stringent conditions, and encoding a protein having an enzyme activity that catalyzes a reaction of cis-chain elongation of isoprenoid compounds;
[F3] a DNA comprising a base sequence having 80% or more sequence identity with the base sequence of base numbers 1 to 873 represented by SEQ ID NO: 16, and encoding a protein having an enzyme activity that catalyzes a reaction of cis-chain elongation of isoprenoid compounds;
[F4] a DNA comprising the base sequence of base numbers 1 to 855 represented by SEQ ID NO: 66;
[F5] a DNA hybridizing to a DNA comprising a base sequence complementary to the base sequence of base numbers 1 to 855 represented by SEQ ID NO: 66 under stringent conditions, and encoding a protein having an enzyme activity that catalyzes a reaction of cis-chain elongation of isoprenoid compounds; and
[F6] a DNA comprising a base sequence having 80% or more sequence identity with the base sequence of base numbers 1 to 855 represented by SEQ ID NO: 66, and encoding a protein having an enzyme activity that catalyzes a reaction of cis-chain elongation of isoprenoid compounds.

The gene encoding Small Rubber Particle Protein may preferably comprise any one of the following DNAs:

[G1] a DNA comprising the base sequence of base numbers 1 to 615 represented by SEQ ID NO: 18;
[G2] a DNA hybridizing to a DNA comprising a base sequence complementary to the base sequence of base numbers 1 to 615 represented by SEQ ID NO: 18 under stringent conditions, and encoding a rubber particle-associated protein which is associated with rubber particles in latex; and
[G3] a DNA comprising a base sequence having 80% or more sequence identity with the base sequence of base numbers 1 to 615 represented by SEQ ID NO: 18, and encoding a rubber particle-associated protein which is associated with rubber particles in latex.

The gene encoding Rubber Elongation Factor may preferably comprise any one of the following DNAs:

[H1] a DNA comprising the base sequence of base numbers 1 to 417 represented by SEQ ID NO: 60;
[H2] a DNA hybridizing to a DNA comprising a base sequence complementary to the base sequence of base numbers 1 to 417 represented by SEQ ID NO: 60 under stringent conditions, and encoding a rubber particle-associated protein which is associated with rubber particles in latex; and
[H3] a DNA comprising a base sequence having 80% or more sequence identity with the base sequence of base numbers 1 to 417 represented by SEQ ID NO: 60, and encoding a rubber particle-associated protein which is associated with rubber particles in latex.

As used herein, the term “hybridizing” is as described above. Also, the stringent conditions are as described above.

Moreover, like the DNA capable of hybridization under stringent conditions described above, it is known that some protein-encoding genes with base sequences having a certain sequence identity with the original base sequence have similar enzyme activity. In order to maintain the enzyme activity, the sequence identity with the base sequence of base numbers 1 to 1029 represented by SEQ ID NO: 2 is at least 80% or more, preferably 90% or more, more preferably 95% or more, further preferably 98% or more, particularly preferably 99% or more. The same range of the sequence identity as described above applies to the base sequences represented by SEQ ID NOs: 4, 6 to 9, 14, 16, 18, 60, and 66.

Whether a DNA hybridizing to the above-described DNA under stringent conditions or a DNA having 80% or more sequence identity with the above-described DNA encodes a protein having a predetermined function such as enzyme activity may be determined by conventional techniques, such as by expressing a target protein in a transformant prepared by introducing a gene encoding the target protein into Escherichia coli or the like, and determining the presence or absence of the function of the target protein by the corresponding activity measurement method.

Also, conventional techniques may be employed to identify the base sequence of a gene encoding the protein or the amino acid sequence of the protein. For example, the whole length base sequence or amino acid sequence is identified by extracting total RNA from a growing plant, optionally purifying the mRNA, and synthesizing a cDNA by a reverse transcription reaction; then designing degenerate primers based on the amino acid sequence of a known protein corresponding to the target protein, partially amplifying a DNA fragment by RT-PCR, and partially identifying the sequence; and then performing the RACE method or the like. The RACE method (Rapid Amplification of cDNA Ends method) refers to a method in which, when the base sequence of a cDNA is partially known, PCR is performed based on the base sequence information of such a known region to clone an unknown region extending to the cDNA terminal, and is capable of cloning the whole length cDNA by PCR without preparing a cDNA library.

The degenerate primer may preferably be prepared from a plant-derived sequence having a highly similar sequence part to the target protein.

In the case where the base sequence encoding the protein is known, it is possible to identify the whole length base sequence or amino acid sequence by designing a primer including an initiation codon and a primer including a termination codon using the known base sequence and then performing RT-PCR using a synthesized cDNA as a template.

### (Protein)

A specific example of the farnesyl diphosphate synthase may be [b1] described below. The protein designated by [b1] is a protein encoded by the above-described DNA designated by [B1]:

[b1] a protein comprising the amino acid sequence of amino acid numbers 1 to 342 represented by SEQ ID NO: 3.

A specific example of the geranylgeranyl diphosphate synthase may be [c1] described below. The protein designated by [c1] is a protein encoded by the above-described DNA designated by [C1]:

[c1] a protein comprising the amino acid sequence of amino acid numbers 1 to 370 represented by SEQ ID NO: 5.

A specific example of the 3-hydroxy-3-methylglutaryl CoA reductase may be any one of [d1] to [d4] described below. The proteins designated by [d1] to [d4] are proteins encoded by the above-described DNAs designated by [D1], [D4], [D7], and [D10], respectively:

[d1] a protein comprising the amino acid sequence of amino acid numbers 1 to 575 represented by SEQ ID NO: 10;
[d2] a protein comprising the amino acid sequence of amino acid numbers 1 to 586 represented by SEQ ID NO: 11;
[d3] a protein comprising the amino acid sequence of amino acid numbers 1 to 606 represented by SEQ ID NO: 12; and
[d4] a protein comprising the amino acid sequence of amino acid numbers 1 to 526 represented by SEQ ID NO: 13.

A specific example of the isopentenyl diphosphate isomerase may be [e1] described below. The protein designated by [e1] is a protein encoded by the above-described DNA designated by [E1]:

[e1] a protein comprising the amino acid sequence of amino acid numbers 1 to 234 represented by SEQ ID NO: 15.

A specific example of the cis-prenyltransferase may be [f1] or [f4] described below. The proteins designated by [f1] and [f4] are proteins encoded by the above-described DNAs designated by [F1] and [F4], respectively:

[f1] a protein comprising the amino acid sequence of amino acid numbers 1 to 290 represented by SEQ ID NO: 17; and
[f4] a protein comprising the amino acid sequence of amino acid numbers 1 to 284 represented by SEQ ID NO: 67.

A specific example of the Small Rubber Particle Protein may be [g1] described below. The protein designated by [g1] is a protein encoded by the above-described DNA designated by [G1];

[g1] a protein comprising the amino acid sequence of amino acid numbers 1 to 204 represented by SEQ ID NO: 19.

A specific example of the Rubber Elongation Factor may be [h1] described below. The protein designated by [h1] is a protein encoded by the above-described DNA designated by [H1]:

[h1] a protein comprising the amino acid sequence of amino acid numbers 1 to 138 represented by SEQ ID NO: 61.

It is known that some proteins having one or more amino acid substitutions, deletions, insertions, or additions relative to the original amino acid sequence have the inherent function. Thus, specific examples of the above-described proteins also include the following [b2], [c2], [d5], [d6], [d7], [d8], [e2], [f2], [f5], [g2], and [h2]:

[b2] a protein comprising an amino acid sequence containing one or more amino acid substitutions, deletions, insertions, and/or additions relative to the amino acid sequence of amino acid numbers 1 to 342 represented by SEQ ID NO: 3, and having an enzyme activity that catalyzes a reaction using isopentenyl diphosphate and dimethylallyl diphosphate as substrates or a reaction using isopentenyl diphosphate and geranyl diphosphate as substrates;
[c2] a protein comprising an amino acid sequence containing one or more amino acid substitutions, deletions, insertions, and/or additions relative to the amino acid sequence of amino acid numbers 1 to 370 represented by SEQ ID NO: 5, and having an enzyme activity that catalyzes a reaction using isopentenyl diphosphate and dimethylallyl diphosphate as substrates, a reaction using isopentenyl diphosphate and geranyl diphosphate as substrates, or a reaction using isopentenyl diphosphate and farnesyl diphosphate as substrates;
[d5] a protein comprising an amino acid sequence containing one or more amino acid substitutions, deletions, insertions, and/or additions relative to the amino acid sequence of amino acid numbers 1 to 575 represented by SEQ ID NO: 10, and having an enzyme activity that catalyzes a reaction of reduction of 3-hydroxy-3-methylglutaryl CoA;
[d6] a protein comprising an amino acid sequence containing one or more amino acid substitutions, deletions, insertions, and/or additions relative to the amino acid sequence of amino acid numbers 1 to 586 represented by SEQ ID NO: 11, and having an enzyme activity that catalyzes a reaction of reduction of 3-hydroxy-3-methylglutaryl CoA;
[d7] a protein comprising an amino acid sequence containing one or more amino acid substitutions, deletions, insertions, and/or additions relative to the amino acid sequence of amino acid numbers 1 to 606 represented by SEQ ID NO: 12, and having an enzyme activity that catalyzes a reaction of reduction of 3-hydroxy-3-methylglutaryl CoA;
[d8] a protein comprising an amino acid sequence containing one or more amino acid substitutions, deletions, insertions, and/or additions relative to the amino acid sequence of amino acid numbers 1 to 526 represented by SEQ ID NO: 13, and having an enzyme activity that catalyzes a reaction of reduction of 3-hydroxy-3-methylglutaryl CoA;
[e2] a protein comprising an amino acid sequence containing one or more amino acid substitutions, deletions, insertions, and/or additions relative to the amino acid sequence of amino acid numbers 1 to 234 represented by SEQ ID NO: 15, and having an enzyme activity that catalyzes a reaction of isomerization of isopentenyl diphosphate or dimethylallyl diphosphate;
[f2] a protein comprising an amino acid sequence containing one or more amino acid substitutions, deletions, insertions, and/or additions relative to the amino acid sequence of amino acid numbers 1 to 290 represented by SEQ ID NO: 17, and having an enzyme activity that catalyzes a reaction of cis-chain elongation of isoprenoid compounds;
[f5] a protein comprising an amino acid sequence containing one or more amino acid substitutions, deletions, insertions, and/or additions relative to the amino acid sequence of amino acid numbers 1 to 284 represented by SEQ ID NO: 67, and having an enzyme activity that catalyzes a reaction of cis-chain elongation of isoprenoid compounds;
[g2] a rubber particle-associated protein comprising an amino acid sequence containing one or more amino acid substitutions, deletions, insertions, and/or additions relative to the amino acid sequence of amino acid numbers 1 to 204 represented by SEQ ID NO: 19, and being associated with rubber particles in latex; and
[h2] a rubber particle-associated protein comprising an amino acid sequence containing one or more amino acid substitutions, deletions, insertions, and/or additions relative to the amino acid sequence of amino acid numbers 1 to 138 represented by SEQ ID NO: 61, and being associated with rubber particles in latex.

In order to maintain the enzyme activity, preferred is an amino acid sequence containing one or more, more preferably 1 to 68, further preferably 1 to 51, still further preferably 1 to 34, particularly preferably 1 to 17, most preferably 1 to 7, yet most preferably 1 to 3 amino acid substitutions, deletions, insertions, and/or additions relative to the amino acid sequence represented by SEQ ID NO: 3.

Also, in order to maintain the enzyme activity, preferred is an amino acid sequence containing one or more, more preferably 1 to 74, further preferably 1 to 56, still further preferably 1 to 37, particularly preferably 1 to 19, most preferably 1 to 7, yet most preferably 1 to 4 amino acid substitutions, deletions, insertions, and/or additions relative to the amino acid sequence represented by SEQ ID NO: 5.

Also, in order to maintain the enzyme activity, preferred is an amino acid sequence containing one or more, more preferably 1 to 115, further preferably 1 to 86, still further preferably 1 to 58, particularly preferably 1 to 29, most preferably 1 to 12, yet most preferably 1 to 6 amino acid substitutions, deletions, insertions, and/or additions relative to the amino acid sequence represented by SEQ ID NO: 10.

Also, in order to maintain the enzyme activity, preferred is an amino acid sequence containing one or more, more preferably 1 to 117, further preferably 1 to 88, still further preferably 1 to 59, particularly preferably 1 to 29, most preferably 1 to 12, yet most preferably 1 to 6 amino acid substitutions, deletions, insertions, and/or additions relative to the amino acid sequence represented by SEQ ID NO: 11.

Also, in order to maintain the enzyme activity, preferred is an amino acid sequence containing one or more, more preferably 1 to 121, further preferably 1 to 91, still further preferably 1 to 61, particularly preferably 1 to 30, most preferably 1 to 12, yet most preferably 1 to 6 amino acid substitutions, deletions, insertions, and/or additions relative to the amino acid sequence represented by SEQ ID NO: 12.

Also, in order to maintain the enzyme activity, preferred is an amino acid sequence containing one or more, more preferably 1 to 105, further preferably 1 to 79, still further preferably 1 to 53, particularly preferably 1 to 26, most preferably 1 to 11, yet most preferably 1 to 5 amino acid substitutions, deletions, insertions, and/or additions relative to the amino acid sequence represented by SEQ ID NO: 13.

Also, in order to maintain the enzyme activity, preferred is an amino acid sequence containing one or more, more preferably 1 to 47, further preferably 1 to 35, still further preferably 1 to 23, particularly preferably 1 to 12, most preferably 1 to 5, yet most preferably 1 to 2 amino acid substitutions, deletions, insertions, and/or additions relative to the amino acid sequence represented by SEQ ID NO: 15.

Also, in order to maintain the enzyme activity, preferred is an amino acid sequence containing one or more, more preferably 1 to 58, further preferably 1 to 44, still further preferably 1 to 29, particularly preferably 1 to 15, most preferably 1 to 6, yet most preferably 1 to 3 amino acid substitutions, deletions, insertions, and/or additions relative to the amino acid sequence represented by SEQ ID NO: 17.

Also, in order to maintain the enzyme activity, preferred is an amino acid sequence containing one or more, more preferably 1 to 57, further preferably 1 to 43, still further preferably 1 to 28, particularly preferably 1 to 14, most preferably 1 to 6, yet most preferably 1 to 3 amino acid substitutions, deletions, insertions, and/or additions relative to the amino acid sequence represented by SEQ ID NO: 67.

Also, in order to maintain the function as SRPP, i.e., the function of being associated with rubber particles in latex, preferred is an amino acid sequence containing one or more, more preferably 1 to 41, further preferably 1 to 31, still further preferably 1 to 20, particularly preferably 1 to 10, most preferably 1 to 4, yet most preferably 1 to 2 amino acid substitutions, deletions, insertions, and/or additions relative to the amino acid sequence represented by SEQ ID NO: 19.

Also, in order to maintain the function as REF, i.e., the function of being associated with rubber particles in latex, preferred is an amino acid sequence containing one or more, more preferably 1 to 28, further preferably 1 to 21, still further preferably 1 to 14, particularly preferably 1 to 7, most preferably 1 to 3, yet most preferably 1 amino acid substitutions, deletions, insertions, and/or additions relative to the amino acid sequence represented by SEQ ID NO: 61.

Among other amino acid substitutions, conservative substitutions are preferred. Specific examples include substitutions within each of the following groups in the parentheses: (glycine, alanine), (valine, isoleucine, leucine), (aspartic acid, glutamic acid), (asparagine, glutamine), (serine, threonine), (lysine, arginine), (phenylalanine, tyrosine), and the like.

It is also known that some proteins with amino acid sequences having high sequence identity with the original amino acid sequence also have similar function. Thus, specific examples of the above-described proteins also include the following [b3], [c3], [d9], [d10], [d11], [d12], [e3], [f3], [f6], [g3], and [h3]:

[b3] a protein comprising an amino acid sequence having 80% or more sequence identity with the amino acid sequence of amino acid numbers 1 to 342 represented by SEQ ID NO: 3, and having an enzyme activity that catalyzes a reaction using isopentenyl diphosphate and dimethylallyl diphosphate as substrates or a reaction using isopentenyl diphosphate and geranyl diphosphate as substrates;
[c3] a protein comprising an amino acid sequence having 80% or more sequence identity with the amino acid sequence of amino acid numbers 1 to 370 represented by SEQ ID NO: 5, and having an enzyme activity that catalyzes a reaction using isopentenyl diphosphate and dimethylallyl diphosphate as substrates, a reaction using isopentenyl diphosphate and geranyl diphosphate as substrates, or a reaction using isopentenyl diphosphate and farnesyl diphosphate as substrates;
[d9] a protein comprising an amino acid sequence having 80% or more sequence identity with the amino acid sequence of amino acid numbers 1 to 575 represented by SEQ ID NO: 10, and having an enzyme activity that catalyzes a reaction of reduction of 3-hydroxy-3-methylglutaryl CoA;
[d10] a protein comprising an amino acid sequence having 80% or more sequence identity with the amino acid sequence of amino acid numbers 1 to 586 represented by SEQ ID NO: 11, and having an enzyme activity that catalyzes a reaction of reduction of 3-hydroxy-3-methylglutaryl CoA;
[d11] a protein comprising an amino acid sequence having 80% or more sequence identity with the amino acid sequence of amino acid numbers 1 to 606 represented by SEQ ID NO: 12, and having an enzyme activity that catalyzes a reaction of reduction of 3-hydroxy-3-methylglutaryl CoA;
[d12] a protein comprising an amino acid sequence having 80% or more sequence identity with the amino acid sequence of amino acid numbers 1 to 526 represented by SEQ ID NO: 13, and having an enzyme activity that catalyzes a reaction of reduction of 3-hydroxy-3-methylglutaryl CoA;
[e3] a protein comprising an amino acid sequence having 80% or more sequence identity with the amino acid sequence of amino acid numbers 1 to 234 represented by SEQ ID NO: 15, and having an enzyme activity that catalyzes a reaction of isomerization of isopentenyl diphosphate or dimethylallyl diphosphate;
[f3] a protein comprising an amino acid sequence having 80% or more sequence identity with the amino acid sequence of amino acid numbers 1 to 290 represented by SEQ ID NO: 17, and having an enzyme activity that catalyzes a reaction of cis-chain elongation of isoprenoid compounds;
[f6] a protein comprising an amino acid sequence having 80% or more sequence identity with the amino acid sequence of amino acid numbers 1 to 284 represented by SEQ ID NO: 67, and having an enzyme activity that catalyzes a reaction of cis-chain elongation of isoprenoid compounds;
[g3] a rubber particle-associated protein comprising an amino acid sequence having 80% or more sequence identity with the amino acid sequence of amino acid numbers 1 to 204 represented by SEQ ID NO: 19, and being associated with rubber particles in latex; and
[h3] a rubber particle-associated protein comprising an amino acid sequence having 80% or more sequence identity with the amino acid sequence of amino acid numbers 1 to 138 represented by SEQ ID NO: 61, and being associated with rubber particles in latex.

In order to maintain the original function of the protein, e.g., the enzyme activity, the sequence identity with the amino acid sequence represented by any one of SEQ ID NOs: 3, 5, 10, 11, 12, 13, 15, 17, 19, 61, and 67 is 80% or more, preferably 85% or more, more preferably 90% or more, further preferably 95% or more, particularly preferably 98% or more, most preferably 99% or more.

Whether it is a protein having a predetermined function such as enzyme activity may be determined by conventional techniques, such as by expressing a target protein in a transformant prepared by introducing a gene encoding the target protein into Escherichia coli or the like, and determining the presence or absence of the function of the target protein by the corresponding activity measurement method.

### (Transformant)

By introducing the vector of the present invention (vector comprising a base sequence in which a gene encoding a protein involved in polyisoprenoid biosynthesis is functionally linked to a promoter of a gene encoding HEV 2.1) into a plant, an organism (transformant) which has been transformed to express the predetermined protein involved in polyisoprenoid biosynthesis specifically in laticifers can be obtained. In the transformant, due to the laticifer-specific expression of the predetermined protein involved in polyisoprenoid biosynthesis, the predetermined function, e.g. enzyme activity of the protein is newly enhanced in the laticifers in the plant having the vector of the present invention introduced therein to enhance a part of the polyisoprenoid biosynthesis pathway, resulting in an improved polyisoprenoid production in the plant.

The host to be used for the transformant is not particularly limited as long as the host is a plant and may preferably be a plant capable of producing a polyisoprenoid. Examples include plants of the genus Hevea, such as Hevea brasiliensis (para rubber tree); plants of the genus Sonchus, such as Sonchus oleraceus (common sowthistle), Sonchus asper, and Sonchus brachyotus; plants of the genus Solidago, such as Solidago altissima (Canada goldenrod), Solidago virgaurea subsp. asiatica, Solidago virgaurea subsp. leipcarpa, Solidago virgaurea subsp. leipc arpaf. paludosa, Solidago virgaurea subsp. gigantea, and Solidago gigantea Ait. var. leiophylla Fernald; plants of the genus Helianthus, such as Helianthus annuus (sunflower), Helianthus argophyllus, Helianthus atrorubens, Helianthus debilis, Helianthus decapetalus, and Helianthus giganteus; plants of the genus Taraxacum, such as Taraxacum, Taraxacum venustum H. Koidz, Taraxacum hondoense Nakai, Taraxacum platycarpum Dahlst, Taraxacum japonicum, Taraxacum officinale Weber, and Taraxacum kok-saghyz(Russian dandelion); plants of the genus Ficus, such as Ficus carica, Ficus elastica, Ficus pumila L., Ficus erecta Thunb., Ficus ampelas Burm. f., Ficus benguetensis Merr, Ficus irisana Elm., Ficus microcarpa L.f., Ficus septica Burm. f., and Ficus benghalensis; plants of the genus Parthenium, such as Parthenium argentatum (guayule), Parthenium hysterophorus, and Parthenium hysterophorus; and Lactuca serriola (lettuce) and Indian banyan. Among them, the plant may more preferably be at least one selected from the group consisting of plants of the genera Hevea, Sonchus, Taraxacum, and Parhenium, particularly preferably plants of the genus Hevea, most preferably Hevea brasiliensis (para rubber tree).

The vector of the present invention can be introduced by any method that allows the DNA to be introduced into plant cells. Examples include a method using Agrobacterium (JP S59-140885 A, JP S60-70080 A, WO94/00977, which are incorporated herein by reference), electroporation (JP S60-251887 A, incorporated herein by reference), and a method using a particle gun (gene gun) (JP 2606856 B, JP 2517813 B, which are incorporated herein by reference). Among them, the method using Agrobacterium (Agrobacterium method) may preferably be used to introduce the vector of the present invention into a plant to prepare a transformant. In this case, a transformant into which a predetermined gene contained in the vector of the present invention has been introduced can be prepared by introducing the vector of the present invention into a bacterium of the genus Agrobacterium and culturing and proliferating the Agrobacterium by an ordinary method (for example, shake culture in YEB medium for 10 to 30 hours at a culture temperature of 20° C. to 35° C.), followed by infecting a callus, plant tissue slice, or plantlet with the Agrobacterium.

The Agrobacterium containing the vector of the present invention can be prepared by conventional techniques, such as by inserting the base sequence of a promoter of a gene encoding HEV2.1, the base sequence of a gene encoding a protein involved in polyisoprenoid biosynthesis, and the like into a plasmid capable of homologous recombination with the T-DNA region of the Ti plasmid of Agrobacterium bacteria to prepare a gene recombinant vector as the vector of the present invention and introducing the vector into an Agrobacterium, or by inserting the base sequence of a promoter of a gene encoding HEV2.1, the base sequence of a gene encoding a protein involved in polyisoprenoid biosynthesis, and the like into the above-described binary vector to prepare a gene recombinant binary vector as the vector of the present invention and introducing the vector into an Agrobacterium.

The Agrobacterium may be Agrobacterium tumefaciens (e.g. C58, LBA4404, EHA101, EHA105, C58C1RifR, GV3101).

The transformant (transgenic plant cell) can be obtained by the above-described methods and so forth.

The present invention also provides a transgenic plant into which the vector of the present invention has been introduced. The transgenic plant is not particularly limited as long as the plant has transgenic plant cells. It conceptually includes, for example, not only transgenic plant cells obtained by the above-described methods but also all their progeny or clones and even progeny plants obtained by passaging these cells. Once transgenic plant cells into which the base sequence of a promoter of a gene encoding HEV2.1 and the base sequence of a gene encoding a protein involved in polyisoprenoid biosynthesis in the vector of the present invention have been introduced in the genome are obtained, progeny or clones can be obtained from the transgenic plant cells by sexual or asexual reproduction, tissue culture, cell culture, cell fusion, or the like. Further, the transgenic plant cells, or progeny or clones thereof may be used to obtain reproductive materials (e.g. seeds, fruits, cuttings, stem tubers, root tubers, shoots, adventitious buds, adventitious embryos, calluses, protoplasts), which can then be used to produce the plant on a large scale.

Techniques to regenerate plants (transgenic plants) from transgenic plant cells are already known; for example, Doi et al. disclose techniques for eucalyptus (JP 2000-316403 A), Fujimura et al. disclose techniques for rice (Fujimura et al., (1995), Plant Tissue Culture Lett., vol. 2: p 74-), Shillito et al. disclose techniques for corn (Shillito et al., (1989), Bio/Technology, vol. 7: p 581-), Visser et al. disclose techniques for potato (Visser et al., (1989), Theor. Appl. Genet., vol. 78: p 589-), and Akama et al. disclose techniques for Arabidopsis thaliana (Akama et al., (1992), Plant Cell Rep., vol. 12: p 7-)(all the above documents are incorporated herein by reference). Those skilled in the art can regenerate plants from transgenic plant cells according to these documents.

An example of the method for preparing the transgenic plant of the present invention will be specifically described below.

An example of the method for preparing the transgenic plant of the present invention may include: an infection step of infecting a callus obtained by culturing a plant-derived tissue under callus-inducing conditions (induction step) for 5 to 9 weeks with an Agrobacterium containing the vector of the present invention; a selective culture step of selectively growing the callus having the vector introduced therein; and a step of inducing an adventitious embryo from the callus (regeneration-inducing step). With such a preparation method, a transgenic plant can be prepared by preparing transgenic plant cells (transformed callus) by the infection step and the selective culture step, then inducing an adventitious embryo from the callus by the regeneration-inducing step, and culturing the adventitious embryo to regenerate a plant from the callus. More specifically, a plant may be regenerated from the callus by inducing an adventitious embryo from the callus, culturing the adventitious embryo to form a shoot, and culturing the shoot.

More specifically, the method may preferably include: an infection step of infecting a callus obtained by culturing a plant-derived tissue under callus-inducing conditions for 5 to 9 weeks with an Agrobacterium containing the vector of the present invention; a selective culture step of selectively growing the callus having the vector introduced therein; a regeneration-inducing step of culturing the callus in a regeneration-inducing medium to form an adventitious embryo and a shoot; and a rooting step of culturing the shoot in a rooting medium to root it, and more preferably include: an infection step of infecting a callus obtained by culturing a plant-derived tissue under callus-inducing conditions for 5 to 9 weeks with an Agrobacterium containing the vector of the present invention; a selective culture step of selectively growing the callus having the vector introduced therein; a regeneration-inducing step of culturing the callus in a regeneration-inducing medium to form an adventitious embryo and a shoot; an elongation step of culturing the formed shoot in an elongation medium to elongate it; and a rooting step of culturing the elongated shoot in a rooting medium to root it.

Hereinafter, the steps of the method for preparing the transgenic plant of the present invention will be described.

### (Induction Step)

First, a method for preparing a callus (induction step) will be described.

In the induction step, a callus is induced, for example, by culturing a tissue slice (tissue) of a plant in an induction medium containing a plant growth hormone and a carbon source.

The tissue slice may preferably be at least one selected from the group consisting of a leaf, a stem, a root, a bud, a petal, a cotyledon, an anther, and a seed without particular limitation thereto. Among them, it may preferably a leaf or a stem.

In the induction step, the surface of the plant tissue slice is first washed. When an internal tissue of a plant is used as the tissue slice, it may for example be washed using a cleanser or in water containing about 0.1% of a surfactant. When a leaf or the like is used, the surface may be washed using a soft sponge.

Subsequently, the tissue slice is disinfected or sterilized. The disinfection or sterilization may be conducted using a known disinfectant or sterilizer, preferably ethanol, benzalkonium chloride, or aqueous sodium hypochlorite.

Next, callus induction is carried out by culturing the disinfected or sterilized tissue slice in an induction medium containing a plant growth hormone and a carbon source. The induction medium may be either a liquid or a solid, but is preferably a solid medium since callus formation can be facilitated by placing and culturing the tissue slice on the medium. When the induction medium is a liquid medium, static culture or shake culture may be performed.

Examples of the plant growth hormone include auxin plant hormones and/or cytokinin plant hormones.

Examples of auxin plant hormones include 2,4-dichlorophenoxyacetic acid (2,4-D), naphthaleneacetic acid, indolebutyric acid, indoleacetic acid, indolepropionic acid, chlorophenoxyacetic acid, naphthoxyacetic acid, phenylacetic acid, 2,4,5-trichlorophenoxyacetic acid, p-chlorophenoxyacetic acid, 2-methyl-4-chlorophenoxyacetic acid, 4-fluorophenoxyacetic acid, 2-methoxy-3,6-dichlorobenzoic acid, 2-phenyl acid, picloram, and picolinic acid. Among these, 2,4-dichlorophenoxyacetic acid, naphthaleneacetic acid, and indolebutyric acid are preferred, and 2,4-dichlorophenoxyacetic acid is more preferred.

Examples of cytokinin plant hormones include benzyladenine, kinetin (KI), zeatin, benzylaminopurine, isopentenyl aminopurine, thidiazuron, isopentenyl adenine, zeatin riboside, and dihydrozeatin. Among these, benzyladenine, kinetin, and zeatin are preferred, and kinetin is more preferred.

The carbon source is not particularly limited, and examples include sugars such as sucrose, glucose, trehalose, fructose, lactose, galactose, xylose, allose, talose, gulose, altrose, mannose, idose, arabinose, apiose, and maltose. Also, sugar alcohols such as erythritol, xylitol, mannitol, sorbitol, lactitol, and the like may be used. Among them, sucrose is preferred.

The induction medium may be any of the following base media supplemented with the plant growth hormone: basal media such as White's medium (disclosed on pages 20 to 36 of Shokubutsu Saibo Kogaku Nyumon (Introduction to Plant Cell Engineering), Japan Scientific Societies Press), Heller's medium (Heller R., Bot. Biol. Veg. Paris 14, 1-223 (1953)), SH medium (medium of Schenk and Hildebrandt), MS medium (medium of Murashige and Skoog) (disclosed on pages 20 to 36 of Shokubutsu Saibo Kogaku Nyumon (Introduction to Plant Cell Engineering), Japan Scientific Societies Press), LS medium (medium of Linsmaier and Skoog) (disclosed on pages 20 to 36 of Shokubutsu Saibo Kogaku Nyumon (Introduction to Plant Cell Engineering), Japan Scientific Societies Press), Gamborg medium, B5 medium (disclosed on pages 20 to 36 of Shokubutsu Saibo Kogaku Nyumon (Introduction to Plant Cell Engineering), Japan Scientific Societies Press), MB medium, and WP medium (Woody Plant: for woody plants)(the disclosures of the foregoing documents are incorporated by reference herein), as well as modified basal media obtained by modifying the composition of the basal media, and the like. Among these, MS medium, B5 medium, or WP medium, supplemented with the plant growth hormone is preferred. Moreover, the medium may preferably contain an auxin plant hormone and a cytokinin plant hormone because such a medium is suitable for maintaining the callus and promoting cell division.

The induction medium may contain at least one selected from the group consisting of jasmonic acid and monoterpene compounds.

Examples of monoterpene compounds include D-limonene, α-pinene, β-pinene, 1-menthol, geraniol, carane, pinane, myrcene, ocimene, cosmene, and so forth. Among them, D-limonene or α-pinene is preferred.

To prepare the induction medium as a solid medium, the medium may be made solid using a solidifying agent. Examples of the solidifying agent include, but are not limited to, agar, gellan gum (e.g. Gelrite), agarose, gelatin, and silica gel.

The suitable composition and culture conditions of the induction medium vary depending on the type of plant, and also vary depending on whether the medium is a liquid medium or a solid medium. Typically (particularly in the case of para rubber tree), the induction medium has the following composition.

The nitrogen concentration in the induction medium may preferably be 0 mM or more, more preferably 1×10⁻³ mM or more, further preferably 20 mM or more. The nitrogen concentration may preferably be 100 mM or less, more preferably 70 mM or less.

The concentration of trace inorganic salts in the induction medium may preferably be 0 mM or more, more preferably 1×10⁻³ mM or more. The concentration of trace inorganic salts may preferably be 2 mM or less, more preferably 0.23 mM or less.

In the present specification, the “trace inorganic slats” means inorganic salts to be contained in small amounts in the medium, such as boron, manganese, zinc, copper, molybdenum, chlorine, cobalt, titanium, vanadium, aluminum, or silicon. In other words, the trace inorganic salts do not include major inorganic salts (inorganic slats with which culture is not achievable unless they are contained in large amounts in the medium) such as calcium, magnesium, and potassium. Among the trace inorganic salts, boron, manganese, and zinc are preferred, and the combined concentration of boron, manganese, and zinc may preferably be within the above preferred range of the concentration of trace inorganic salts.

The concentration of carbon source in the induction medium may preferably be 0.1 mass % or more, more preferably 1 mass % or more. The concentration of carbon source may preferably be 10 mass % or less, more preferably 6 mass % or less. In the present specification, the concentration of the carbon source means the concentration of sugars.

The calcium ion concentration in the induction medium may preferably be 0 mM or more, more preferably 1×10⁻⁵ mM or more, further preferably 0.1 mM or more. The calcium ion concentration may preferably be 10 mM or less, more preferably 5 mM or less.

The concentration of auxin plant hormone in the induction medium may preferably be 0 mg/l or more, more preferably 1×10⁻³ mg/l or more, further preferably 1 mg/l or more, particularly preferably 1.5 mg/l or more. The concentration of auxin plant hormone may also preferably be 20 mg/l or less, more preferably 10 mg/l or less, further preferably 3 mg/l or less, particularly preferably 2.5 mg/l or less.

The concentration of cytokinin plant hormone in the induction medium may preferably be 0 mg/l or more, more preferably 1×10⁻³ mg/l or more, further preferably 0.5 mg/l or more, particularly preferably 0.8 mg/l or more. The concentration of cytokinin plant hormone may also preferably be 15 mg/l or less, more preferably 10 mg/l or less, further preferably 3 mg/l or less, particularly preferably 1.5 mg/l or less, most preferably 1.2 mg/l or less.

The concentration of jasmonic acid in the induction medium may preferably be 0 mass % or more, more preferably 1×10⁻⁶ mass % or more. The concentration of jasmonic acid may preferably be 0.5 mass % or less, more preferably 0.3 mass % or less.

The concentration of monoterpene compound in the induction medium may preferably be 0 mass % or more, more preferably 1×10⁻⁶ mass % or more. The concentration of monoterpene compound may preferably be 0.5 mass % or less, more preferably 0.3 mass % or less.

The pH of the induction medium may preferably be 4.0 to 10.0, more preferably 5.0 to 6.5, further preferably 5.6 to 5.8. The culture temperature may preferably be 0° C. to 40° C., more preferably 23° C. to 30° C. The culture may be carried out either in a dark place or a bright place, but the illuminance may preferably be 0 to 100000 lx, more preferably 0 to 0.1 lx.

In the present specification, the pH of solid media means the pH of media supplemented with all the components except the solidifying agent. Moreover, in the present specification, the dark place means an illuminance of 0 to 0.1 lx, and the bright place means an illuminance exceeding 0.1 lx.

When the induction medium is a solid medium, the concentration of solidifying agent in the induction medium may preferably be 0.1 mass % or more, more preferably 0.2 mass % or more. The concentration of solidifying agent may also preferably be 2 mass % or less, more preferably 1.1 mass % or less.

Among the above-described conditions, particularly when the plant is para rubber tree, it is particularly preferable that the auxin plant hormone is 2,4-dichlorophenoxyacetic acid at a concentration of 1.5 to 2.5 mg/l, and the cytokinin plant hormone is kinetin at a concentration of 0.8 to 1.2 mg/l.

As described above, callus induction can be carried out by culturing the disinfected or sterilized tissue slice in the induction medium.

In the method for preparing the transgenic plant of the present invention, for example, a callus obtained by culturing a plant-derived tissue under callus-inducing conditions (the above-described induction medium) for 5 to 9 weeks can be used.

The culture period from the start of culture of the plant-derived tissue under callus-inducing conditions until the infection of an Agrobacterium is not particularly limited and may appropriately be set in view of the type of plant, the type and amount of plant tissue (tissue slice) used as a material for callus induction, the amount of Agrobacterium bacteria used for infection, and so forth. The callus to be used may preferably be obtained by culturing a plant-derived tissue under callus-inducing conditions for 5 to 9 weeks, more preferably obtained by culturing a plant-derived tissue under callus-inducing conditions for 6 to 8 weeks, particularly preferably obtained by culturing a plant-derived tissue under callus-inducing conditions for 8 weeks.

The callus obtained by culturing a plant-derived tissue under callus-inducing conditions for 8 weeks, for example, means the one that is obtained by culturing a plant-derived tissue for 8 weeks after transfer to the callus induction medium (the above-described induction medium).

### (Infection Step)

In the infection step, the callus obtained by culturing a plant-derived tissue under callus-inducing conditions e.g. for 5 to 9 weeks is infected with an Agrobacterium containing the vector of the present invention.

In the infection step, the callus obtained by culturing a plant-derived tissue under callus-inducing conditions e.g. for 5 to 9 weeks (the callus obtained by the induction step) is infected with an Agrobacterium containing the vector of the present invention (as prepared as described above).

The infection step may be carried out in a manner commonly used in the Agrobacterium method. For example, the infection can be attained by immersing the callus in a suspension obtained by suspending an Agrobacterium containing the vector of the present invention in an infection medium. After the immersion, the suspension and the callus may then be separated from each other using a filter paper or the like. During the immersion, the suspension may be left to stand still or may be shaken, but it is preferable to shake the suspension since shaking facilitates the infection of the callus with the Agrobacterium.

The bacterium concentration in the suspension may appropriately be set in view of the type and proliferation activity of the Agrobacterium, the culture period after the callus induction of the callus to be infected, the immersion period, and so forth. For example, it is preferable to bring 6 g of the callus into contact with the Agrobacterium in an amount that corresponds to 10 to 50 mL, preferably 20 to 40 mL, more preferably 25 to 35 mL of an Agrobacterium suspension having an absorbance measured at 600 nm (O. D. 600) of 0.01 to 0.4, preferably 0.05 to 0.2, more preferably 0.08 to 0.12. In this case, the number of Agrobacterium to infect the callus can be optimized to enable efficient preparation of transgenic plant cells.

The coexistence period of the Agrobacterium and the callus in the infection step, i.e., the period during which the Agrobacterium and the callus are in contact with each other may preferably be 0.5 to 60 minutes, more preferably 1 to 40 minutes, further preferably 25 to 35 minutes. In this case, the number of Agrobacterium to infect the callus can be optimized to enable efficient preparation of transgenic plant cells. The coexistence period means, for example, the immersion period if the callus is immersed in an Agrobacterium suspension.

The infection medium for suspending the Agrobacterium may be any of the above-described base media (e.g., basal media or modified basal media obtained by modifying the composition of the basal media) optionally supplemented with a plant growth hormone. Among them, MS medium, LS medium, B5 medium, or WP medium is preferred, and MS medium is more preferred. As the plant growth hormone and the carbon source, those used for the induction medium may suitably be used, but sucrose is further preferred as the carbon source.

The suitable composition of the infection medium varies depending on the type of plant. Typically (particularly in the case of para rubber tree), the infection medium has the following composition.

The concentration of carbon source in the infection medium may preferably be 0.1 mass % or more, more preferably 1 mass % or more, further preferably 2 mass % or more, particularly preferably 3 mass % or more. The concentration of carbon source may also preferably be 10 mass % or less, more preferably 6 mass % or less, further preferably 5 mass % or less.

For better callus conditions, the infection medium may preferably be an amino acid-containing medium. Examples of the amino acid include aspartic acid, glutamine, glutamic acid, asparagine, proline, and so forth, without particular limitation thereto. Among them, aspartic acid or glutamine is preferred, and it is preferable to use aspartic acid and glutamine in combination.

The concentration of amino acid in the infection medium may preferably be 500 mg/l or more, more preferably 700 mg/l or more, further preferably 1000 mg/l or more. The concentration of amino acid may also preferably be 5000 mg/l or less, more preferably 2000 mg/l or less, further preferably 1300 mg/l or less.

When aspartic acid and glutamine are used in combination in the amino acid-containing medium, the concentration of aspartic acid in the infection medium may preferably be 100 to 700 mg/l, more preferably 200 to 500 mg/l, further preferably 250 to 400 mg/l. On the other hand, the concentration of glutamine in the infection medium may preferably be 100 to 1500 mg/l, more preferably 500 to 1200 mg/l, further preferably 700 to 1100 mg/l.

The infection medium may preferably be an acetosyringone-containing medium because the callus can be more readily infected with Agrobacterium. The concentration of acetosyringone in the infection medium may preferably be 0.1 to 30 mg/l, more preferably 1 to 20 mg/l, further preferably 5 to 15 mg/l.

For better callus conditions and better callus growth, the infection medium may preferably be a casamino acid-containing medium. The concentration of casamino acids in the infection medium may preferably be 50 to 600 mg/l, more preferably 100 to 500 mg/l, further preferably 200 to 400 mg/l.

The pH of the infection medium may preferably be 4.0 to 10.0, more preferably 5.0 to 6.0, without particularly limitation thereto. The temperature for infection (temperature of the infection medium) may preferably be 0° C. to 40° C., more preferably 20° C. to 36° C., further preferably 22° C. to 24° C. The infection step may be carried out either in a dark place or a bright place.

As described above, in the infection step, the callus obtained by the induction step can be infected with an Agrobacterium containing the vector of the present invention, for example, by immersing the callus in a suspension obtained by suspending the Agrobacterium in the infection medium.

### (Coculture Step)

In the coculture step, the callus obtained by the infection step (callus infected with the Agrobacterium), for example, is cultured in a coculture medium. In this case, the gene fragment contained in the vector of the present invention introduced into the callus by infection is incorporated into the genes of the plant cells, whereby stable transgenic plant cells can be obtained.

The coculture medium may be either a liquid or a solid, but solid culture is preferred since stable transgenic plant cells can be obtained by placing the callus on the medium. When the coculture medium is a liquid medium, static culture or shake culture may be performed.

The coculture medium may be any of the above-described base media (e.g., basal media or modified basal media obtained by modifying the composition of the basal media) optionally supplemented with a plant growth hormone. Specifically, those mentioned for the infection medium may suitably be used in the same suitable manner.

When the coculture medium is a solid medium, the medium may be made solid using a solidifying agent in the same manner as in the induction medium.

The culture temperature may preferably be 0° C. to 40° C., more preferably 10° C. to 36° C., further preferably 20° C. to 28° C. The culture may be carried out either in a dark place or a bright place, but the culture may preferably be carried out in a dark place, and the illuminance of the dark place may preferably be 0 to 0.1 lx. The culture period may preferably be 2 to 4 days, without particular limitation thereto.

In the case of a solid medium, the concentration of solidifying agent in the coculture medium may preferably be 0.1 mass % or more, more preferably 0.2 mass % or more. The concentration of solidifying agent may preferably be 2 mass % or less, more preferably 1.1 mass % or less, further preferably 0.6 mass % or less.

As described above, in the coculture step, stable transgenic plant cells can be obtained since the gene fragment contained in the vector of the present invention introduced into the callus by infection is incorporated into the genes of the plant cells by culturing the callus obtained by the infection step (callus infected with the Agrobacterium) in the coculture medium. The calluses (mixture of the transformed callus and the non-transformed callus) obtained by the coculture step are used in the subsequent selective culture step.

### (Selective Culture Step)

The selective culture step may be carried out in a manner commonly used in the Agrobacterium method. With this step, the transformed callus and the non-transformed callus can be separated from each other.

In the selective culture step, the calluses (mixture of the transformed callus and the non-transformed callus) obtained by the coculture step are first washed using any of the above-described base media (e.g., basal media or modified basal media obtained by modifying the composition of the basal media) supplemented with carbenicillin to sterilize Agrobacterium. Before the sterilization, the calluses (mixture of the transformed callus and the non-transformed callus) obtained by the coculture step may previously be washed with the above-described base medium (e.g., basal media or modified basal media obtained by modifying the composition of the basal media).

Subsequently, the calluses sterilized with carbenicillin are cultured in a selective culture medium. The culture conditions in the selective culture step are not particularly limited as long as they allow the transgenic plant cells (callus that has acquired the predetermined promoter and the gene encoding the predetermined protein in the vector of the present invention) to be selectively grown.

The selective culture medium may be a liquid or a solid. When the selective culture medium is a liquid medium, static culture or shake culture may be performed.

The selective culture medium may be any of the above-described base media (e.g., basal media or modified basal media obtained by modifying the composition of the basal media) supplemented with a substance corresponding to the marker gene (the marker gene contained in the vector of the present invention). Among them, MS medium, LS medium, B5 medium, or WP medium supplemented with a substance corresponding to the marker gene is preferred, and MS medium supplemented with a substance corresponding to the marker gene is more preferred. Carbenicillin may optionally be added. Also, a plant growth hormone may optionally be added. As the plant growth hormone and the carbon source, those used in the induction medium may suitably be used.

The substance corresponding to the marker gene is not particularly limited, and those skilled in the art can appropriately select the substance according to the marker gene used. When a hygromycin-resistant gene is used as the marker gene, for example, the calluses (carbenicillin-sterilized calluses (mixture of the transformed callus and the non-transformed callus)) are cultured in the medium supplemented with hygromycin, and then the transformed callus can grow in the medium because the hygromycin-resistant gene is also introduced together with the predetermined promoter and the gene encoding the predetermined protein while the non-transformed callus cannot grow in the medium. As described above, the transformed callus can be selectively grown by culturing a mixture of the transformed callus and the non-transformed callus in the medium supplemented with a substance corresponding to the marker gene.

When the selective culture medium is a solid medium, the medium may be made solid using a solidifying agent in the same manner as in the induction medium.

The pH of the selective culture medium may preferably be 5.0 to 7.0, more preferably 5.6 to 6.5, without particular limitation thereto. The culture temperature may preferably be 0° C. to 40° C., more preferably 10° C. to 36° C., further preferably 20° C. to 28° C. The culture may be carried out either in a dark place or a bright place, but the culture may preferably be carried out in a dark place, and the illuminance of the dark place may preferably be 0 to 0.1 lx. The culture period is not particularly limited, but it is preferable to perform subculture every 1 to 4 weeks.

In the case of a solid medium, the concentration of solidifying agent in the selective culture medium may preferably be 0.1 mass % or more, more preferably 0.2 mass % or more. The concentration of solidifying agent may preferably be 2 mass % or less, more preferably 1.1 mass % or less, further preferably 0.6 mass % or less.

As described above, in the selective culture step, the calluses (mixture of the transformed callus and the non-transformed callus) obtained by the coculture step are washed with the carbenicillin-containing solution to sterilize the Agrobacterium. After that, the calluses sterilized with carbenicillin are cultured in the selective culture medium to selectively grow the transformed callus, whereby the transformed callus and the non-transformed callus can be separated from each other.

With the method described above, transgenic plant cells (transformed callus) can be efficiently prepared. Subsequently, the transformed callus is used in the regeneration-inducing step to stably regenerate a plant.

### (Regeneration-Inducing Step)

In the regeneration-inducing step, an adventitious embryo and a shoot are formed by culturing the transformed callus (which may be obtained by proliferating the transformed callus) in a regeneration-inducing medium containing a plant growth hormone and a carbon source. Since it is possible to stably form a shoot by inducing (forming) an adventitious embryo from the callus and culturing the adventitious embryo, the culture conditions in the regeneration-inducing step are not particularly limited as long as they allow an adventitious embryo to be induced from the callus.

In the regeneration-inducing step, an adventitious embryo is induced by culturing the transformed callus in a regeneration-inducing medium. The regeneration-inducing medium may be either a liquid or a solid, but solid culture is preferred since an adventitious embryo can be induced more easily by placing the callus on the medium. When the induction medium is a liquid medium, static culture or shake culture may be performed.

The regeneration-inducing medium may be any of the above-described base media (e.g., basal media or modified basal media obtained by modifying the composition of the basal media) supplemented with a plant growth hormone. Among them, MS medium, LS medium, B5 medium, or WP medium supplemented with a plant growth hormone is preferred, and MS medium supplemented with a plant growth hormone is more preferred. As the plant growth hormone and the carbon source, those used in the induction medium may suitably be used. The medium may preferably contain an auxin plant hormone and a cytokinin plant hormone because such a medium is suitable for inducing an adventitious embryo.

When the regeneration-inducing medium is a solid medium, the medium may be made solid using a solidifying agent in the same manner as in the induction medium.

The suitable composition and culture conditions of the regeneration-inducing medium vary depending on the type of plant, and also vary depending on whether the medium is a liquid medium or a solid medium. Typically (particularly in the case of para rubber tree), the regeneration-inducing medium has the following composition.

The concentration of carbon source in the regeneration-inducing medium may preferably be 0.1 mass % or more, more preferably 1 mass % or more, further preferably 2 mass % or more. The concentration of carbon source may preferably be 10 mass % or less, more preferably 6 mass % or less, further preferably 4 mass % or less.

The concentration of auxin plant hormone in the induction medium may preferably be 0 mg/l or more, more preferably 1×10⁻³ mg/l or more, further preferably 5×10⁻³ mg/l or more. The concentration of auxin plant hormone may preferably be 5 mg/l or less, more preferably 1 mg/l or less, further preferably 0.5 mg/l or less, particularly preferably 0.1 mg/l or less, most preferably 0.03 mg/l or less, yet most preferably 0.01 mg/l or less.

The concentration of cytokinin plant hormone in the induction medium may preferably be 0 mg/l or more, more preferably 1×10⁻³ mg/l or more, further preferably 0.01 mg/l or more, particularly preferably 0.5 mg/l or more, most preferably 0.8 mg/l or more, yet most preferably 1.0 mg/l or more. The concentration of cytokinin plant hormone may preferably be 10 mg/l or less, more preferably 5 mg/l or less, further preferably 2 mg/l or less, particularly preferably 1.5 mg/l or less, most preferably 1.2 mg/l or less. When the concentration of cytokinin plant hormone is within the above-described range, particularly an adventitious embryo can be suitably induced and a shoot can be suitably formed.

The pH of the regeneration-inducing medium may preferably be 4.0 to 10.0, more preferably 5.6 to 6.5, further preferably 5.7 to 5.8, without particular limitation thereto. The culture temperature may preferably be 0° C. to 40° C., more preferably 10° C. to 36° C., further preferably 20° C. to 25° C. The culture may be carried out either in a dark place or a bright place, but the culture may preferably be carried out in a bright place for 10 to 16 hours out of 24 hours, and the illuminance of the bright place may preferably be 2000 to 25000 lx. Regarding the culture period, culture for 1 to 10 months, particularly preferably 3 to 6 months is preferred, without particular limitation thereto. In such a case, it is preferable to perform subculture every 1 to 4 weeks.

In the case of a solid medium, the concentration of solidifying agent in the regeneration-inducing medium may preferably be 0.1 mass % or more, more preferably 0.15 mass % or more. The concentration of solidifying agent may preferably be 2 mass % or less, more preferably 1.1 mass % or less, further preferably 0.6 mass % or less, particularly preferably 0.3 mass % or less.

In the case of para rubber tree, it is preferable that MS medium is used as the base medium for the regeneration-inducing medium and the regeneration-inducing medium has a sucrose concentration of 2 to 4 mass %, a 2,4-dichlorophenoxyacetic acid concentration of 1×10⁻³ to 0.03 mg/l, a kinetin concentration of 0.8 to 1.2 mg/l, and a solidifying agent (gellan gum) concentration of 0.1 to 0.3 mass %.

As described above, in the regeneration-inducing step, an adventitious embryo and a shoot can be formed by culturing the callus in the regeneration-inducing medium. The shoot formed by the regeneration-inducing step is used in the subsequent elongation step. A preferred timing for shifting to the subsequent elongation step is after a shoot has been visually observed and its stable growth has been found. The subsequent elongation step may be omitted to use the shoot formed by the regeneration-inducing step directly in the rooting step.

### (Elongation Step)

In the elongation step, the formed shoot is elongated by culturing the shoot in an elongation medium.

In the elongation step, the shoot formed by the regeneration-inducing step, for example, is elongated by culturing the shoot in an elongation medium. The elongation medium may be either a liquid or a solid, but solid culture is preferred since the shoot can be elongated more easily by placing the shoot on the medium. When the elongation medium is a liquid medium, static culture or shake culture may be performed.

The elongation medium may be any of the above-described basal media or modified basal media obtained by modifying the composition of the basal media, but it may be the same medium as the regeneration-inducing medium because the shoot can be suitably elongated. Among others, the elongation medium may preferably be a medium that does not contain any plant growth hormone, more preferably MS medium that does not contain any plant growth hormone. As the carbon source, those used in the induction medium may suitably be used.

When the elongation medium is a solid medium, the medium may be made solid using solidifying agent in the same manner as in the induction medium.

The suitable culture conditions in the elongation step vary depending on the type of plant and also vary depending on whether the medium is a liquid medium or a solid medium. Typically (particularly in the case of para rubber tree), the following conditions are used.

The pH of the elongation medium may preferably be 4.0 to 10.0, more preferably 5.6 to 6.5, further preferably 5.7 to 5.8, without particular limitation thereto. The culture temperature may preferably be 0° C. to 40° C., more preferably 10° C. to 36° C., further preferably 20° C. to 25° C. The culture may be carried out either in a dark place or a bright place, but the culture may preferably be carried out in a bright place for 10 to 16 hours out of 24 hours, and the illuminance of the bright place may preferably be 2000 to 25000 lx. As for the culture period, culture for 5 to 10 weeks is preferred, without particular limitation thereto.

In the case of a solid medium, the concentration of solidifying agent in the elongation medium may preferably be 0.1 mass % or more, more preferably 0.2 mass % or more. The concentration of solidifying agent may preferably be 2 mass % or less, more preferably 1.1 mass % or less, further preferably 0.6 mass % or less.

As described above, in the elongation step, the formed shoot can be elongated by culturing the short in the elongation medium. Further, not only the elongation of the shoot but also the formation of a new shoot is achieved in the elongation step. The shoot elongated by the elongation step is used in the subsequent rooting step. A preferred timing for shifting to the subsequent rooting step is after the shoot has been elongated to a size of about 2 to 3 cm.

### (Rooting Step)

In the rooting step, the shoot is rooted by culturing in a rooting medium. As the shoot, the shoot elongated by the elongation step may be used, or the shoot formed by the regeneration-inducing step may directly be used.

In the rooting step, the shoot elongated by the elongation step or the shoot formed by the regeneration-inducing step, for example, is rooted by culturing in a rooting medium. The rooting medium may be either a liquid or a solid, but solid culture is preferred since the shoot can be rooted more easily by placing the shoot on the medium. When the rooting medium is a liquid medium, static culture or shake culture may be performed.

The rooting medium may be any of the above-described basal media or modified basal media obtained by modifying the composition of the basal media, but the rooting medium may preferably be a medium that does not contain any plant growth hormone, more preferably ½ MS medium that does not contain any plant growth hormone, because the shoot can be suitably rooted. As the carbon source, those used in the induction medium may suitably be used. The composition of the rooting medium may be the same as that of the elongation medium. Moreover, the rooting step may be omitted when rooting has already occurred in the elongation step.

When the rooting medium is a solid medium, the medium may be made solid using a solidifying agent in the same manner as in the induction medium.

The suitable culture conditions in the rooting step vary depending on the type of plant and also vary depending on whether the medium is a liquid medium or a solid medium. Typically (particularly in the case of para rubber tree), the following conditions are used.

The pH of the rooting medium may preferably be 4.0 to 10.0, more preferably 5.6 to 6.5, further preferably 5.7 to 5.8, without particular limitation thereto. The culture temperature may preferably be 0° C. to 40° C., more preferably 10° C. to 36° C., further preferably 20° C. to 25° C. The culture may be carried out either in a dark place or a bright place, but the culture may preferably be carried out in a bright place for 10 to 16 hours out of 24 hours, and the illuminance of the bright place may preferably be 2000 to 25000 lx. As for the culture period, culture for 4 to 10 weeks is preferred, without particular limitation thereto.

In the case of a solid medium, the concentration of solidifying agent in the rooting medium may preferably be 0.1 mass % or more, more preferably 0.2 mass % or more, further preferably 0.3 mass % or more. The concentration of solidifying agent may preferably be 2 mass % or less, more preferably 1.1 mass % or less, further preferably 0.6 mass % or less.

As described above, in the rooting step, the elongated shoot can be rooted by culturing the shoot in the rooting medium, whereby the rooted shoot (plantlet (transgenic plant)) can be obtained. The plantlet may directly be transplanted to soil, but it may preferably be transferred to and acclimatized in an artificial soil such as vermiculite before transplantation to soil.

In the regenerated plant, the expression of the target protein gene can be confirmed by known techniques. For example, the expression of the target protein may be analyzed by Western blot analysis.

In the present invention, by introducing the vector of the present invention into a plant, the gene encoding a protein involved in polyisoprenoid biosynthesis in the vector is expressed specifically in laticifers, thereby improving polyisoprenoid production in the plant. Specifically, a polyisoprenoid can be produced by culturing the transgenic plant cells obtained by the above-described method, the callus obtained from the transgenic plant cells, the cells redifferentiated from the callus, or the like in an appropriate medium or by growing the transgenic plant regenerated from the transgenic plant cells, the plant obtained from the seeds obtained from the transgenic plant, or the like under appropriate cultivation conditions. Since a part of the polyisoprenoid biosynthesis pathway in laticifers is enhanced by the introduced protein in the transgenic plant of the present invention, the protein (enzyme) can function so as to enhance the amount of the compound biosynthesized in the laticifers and therefore to improve polyisoprenoid productivity in the plant.

In the present specification, the term “polyisoprenoid” is a collective term for polymers having isoprene units (C₅H₈). Examples of the polyisoprenoid include monoterpenes (C₁₀), sesquiterpenes (C₁₅), diterpenes (C₂₀), sesterterpenes (C₂₅), triterpenes (C₃₀), tetraterpenes (C₄₀), natural rubber, farnesyl diphosphate, geranylgeranyl diphosphate, and other polymers. Among them, it may preferably be a polymer having isoprene units with a weight average molecular weight of 1000 or more, more preferably a polymer having isoprene units with a weight average molecular weight of 10000 or more, and further preferably a polymer having isoprene units with a weight average molecular weight of 100000 or more.

The weight average molecular weight can be measured by the method described in Examples.

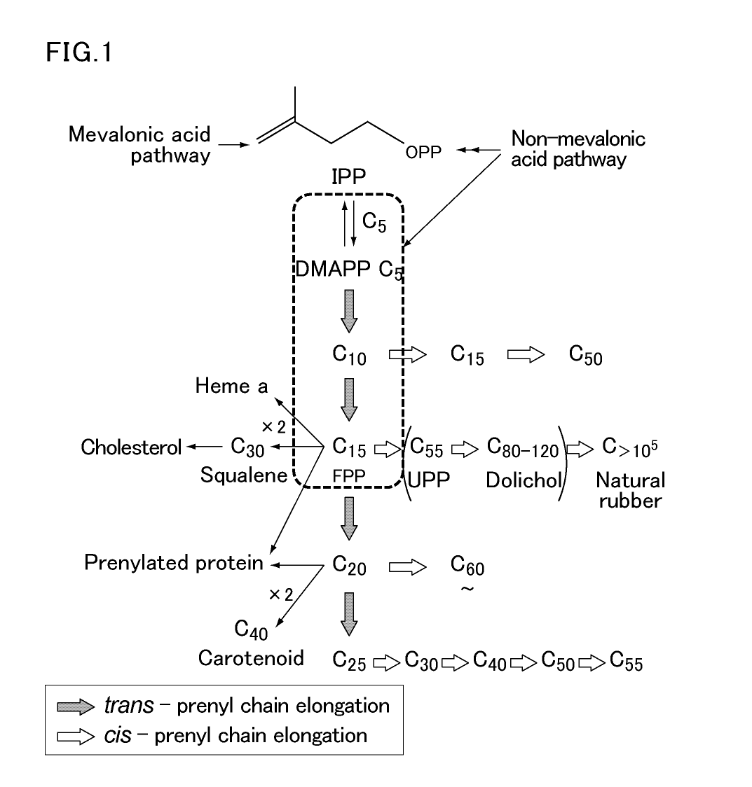

In a polyisoprenoid-producing plant, a polyisoprenoid is biosynthesized via the polyisoprenoid biosynthesis pathway as shown in FIG. 1, in which farnesyl diphosphate synthase is an enzyme that catalyzes the reaction enclosed by the dotted frame in FIG. 1. Accordingly, farnesyl diphosphate synthase activity is increased specifically in laticifers by introducing into a plant a base sequence in which the gene designated by any one of [B1] to [B3] or the gene encoding the protein designated by any one of [b1] to [b3] is functionally linked to a promoter of a gene encoding HEV 2.1. As a result, the amount of farnesyl diphosphate biosynthesized in the laticifers can be enhanced, and therefore polyisoprenoid production in the plant can be improved. Further, it is also possible to increase the weight average molecular weight of polyisoprenoid to be produced. Thus, another aspect of the present invention is a method of increasing the weight average molecular weight of polyisoprenoid to be produced in a plant by introducing into the plant a vector comprising a base sequence in which a gene encoding farnesyl diphosphate synthase is functionally linked to a promoter of a gene encoding HEV 2.1.

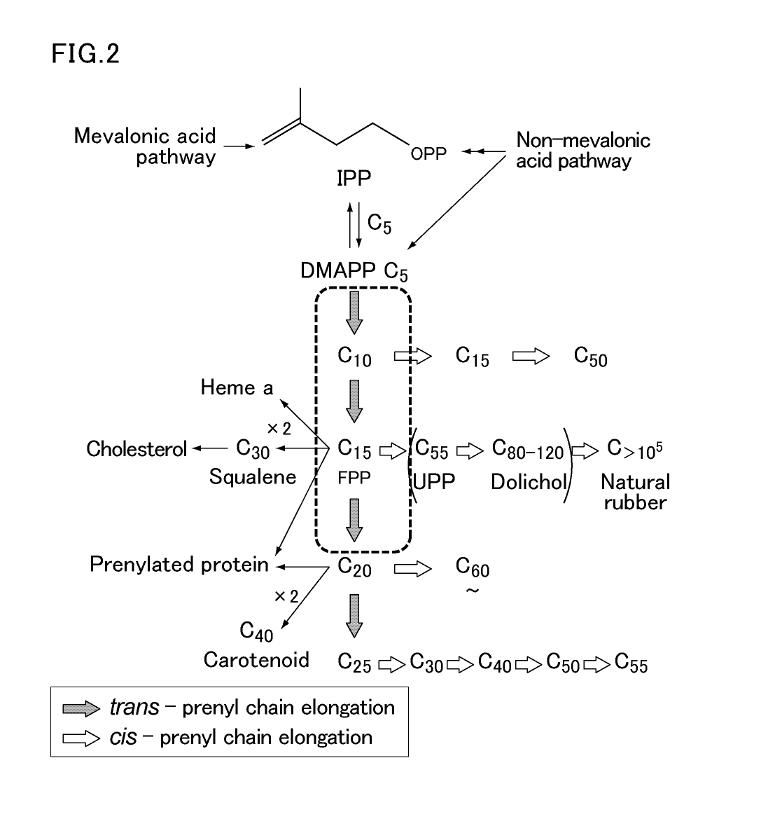

In a polyisoprenoid-producing plant, a polyisoprenoid is biosynthesized via the polyisoprenoid biosynthesis pathway as shown in FIG. 2, in which geranylgeranyl diphosphate synthase is an enzyme that catalyzes the reaction enclosed by the dotted frame in FIG. 2. Accordingly, geranylgeranyl diphosphate synthase activity is increased specifically in laticifers by introducing into a plant a base sequence in which the gene designated by any one of [C1] to [C3] or the gene encoding the protein designated by any one of [c1] to [c3] is functionally linked to a promoter of a gene encoding HEV 2.1. As a result, the amount of geranylgeranyl diphosphate biosynthesized in the laticifers can be enhanced, and therefore polyisoprenoid production can be improved in the plant. Further, it is also possible to increase the weight average molecular weight of polyisoprenoid to be produced. Thus, another aspect of the present invention is a method of increasing the weight average molecular weight of polyisoprenoid to be produced in a plant by introducing into the plant a vector comprising a base sequence in which a gene encoding geranylgeranyl diphosphate synthase is functionally linked to a promoter of a gene encoding HEV 2.1.

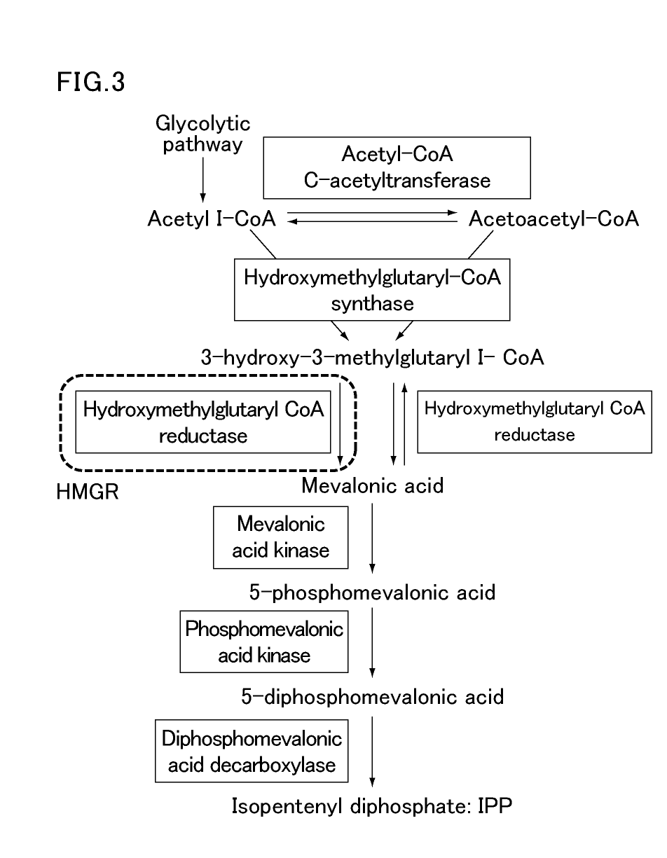

In a polyisoprenoid-producing plant, a polyisoprenoid is biosynthesized via the polyisoprenoid biosynthesis pathway as shown in FIG. 1, and the mevalonic acid pathway located upstream of the pathway is the one as shown in FIG. 3, in which 3-hydroxy-3-methylglutaryl CoA reductase is an enzyme that catalyzes the reaction enclosed by the dotted frame in FIG. 3 and is a rate-limiting enzyme in the mevalonic acid pathway. Accordingly, 3-hydroxy-3-methylglutaryl CoA reductase activity is increased specifically in laticifers by introducing into a plant a base sequence in which the gene designated by any one of [D1] to [D12] or the gene encoding the protein designated by any one of [d1] to [d12] is functionally linked to a promoter of a gene encoding HEV 2.1. As a result, the amount of mevalonic acid biosynthesized in the laticifers can be enhanced, which leads to an improved amount of isopentenyl diphosphate biosynthesized and therefore an improved polyisoprenoid production in the plant. Further, it is also possible to increase the weight average molecular weight of polyisoprenoid to be produced. Thus, another aspect of the present invention is a method of increasing the weight average molecular weight of polyisoprenoid to be produced in a plant by introducing into the plant a vector comprising a base sequence in which a gene encoding 3-hydroxy-3-methylglutaryl CoA reductase is functionally linked to a promoter of a gene encoding HEV 2.1.

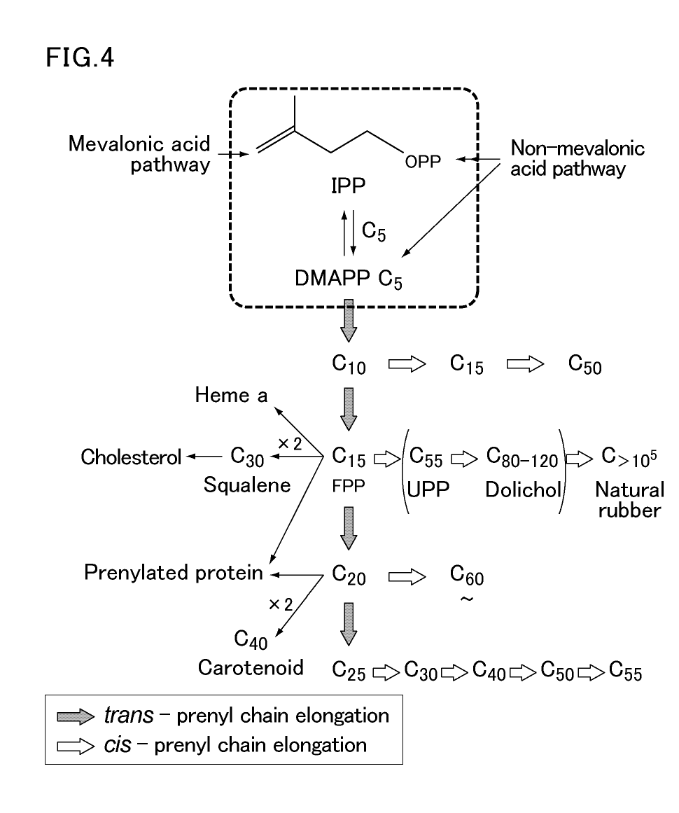

In a polyisoprenoid-producing plant, a polyisoprenoid is biosynthesized via the polyisoprenoid biosynthesis pathway as shown in FIG. 4, in which isopentenyl diphosphate isomerase is an enzyme that catalyzes the reaction enclosed by the dotted frame in FIG. 4. Accordingly, isopentenyl diphosphate isomerase activity is increased specifically in laticifers by introducing into a plant a base sequence in which the gene designated by any one of [E1] to [E3] or the gene encoding the protein designated by any one of [e1] to [e3] is functionally linked to a promoter of a gene encoding HEV 2.1. As a result, the amount of isopentenyl diphosphate or dimethylallyl diphosphate biosynthesized in the laticifers can be enhanced, and therefore polyisoprenoid production can be improved in the plant.

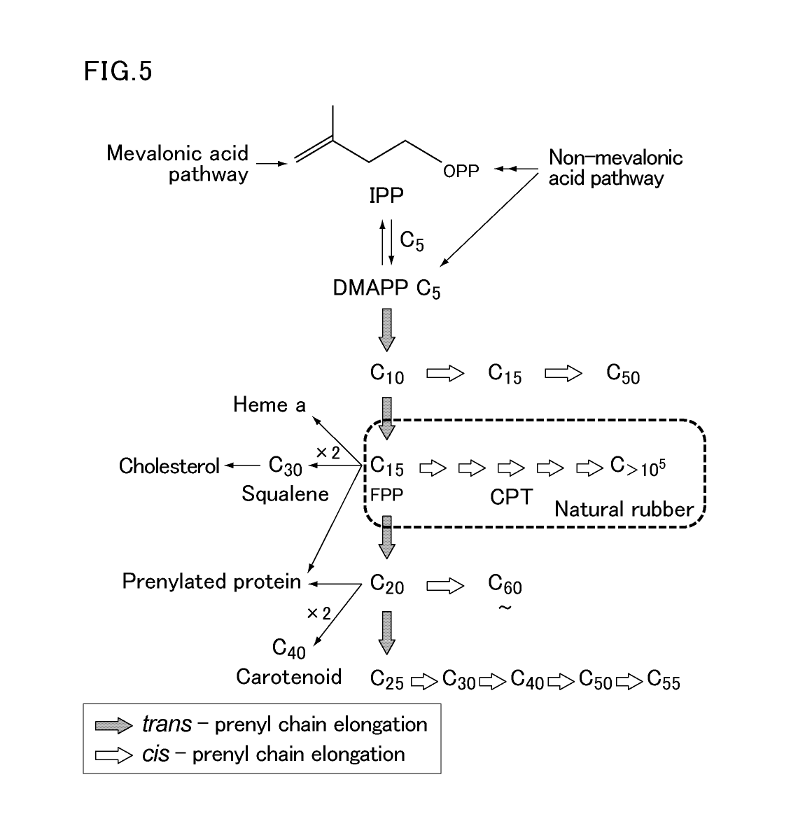

In a polyisoprenoid-producing plant, a polyisoprenoid is biosynthesized via the polyisoprenoid biosynthesis pathway as shown in FIG. 5, in which cis-prenyltransferase is considered to be an enzyme that catalyzes the reaction enclosed by the dotted frame in FIG. 5. Accordingly, cis-prenyltransferase activity is increased specifically in laticifers by introducing into a plant a base sequence in which the gene designated by any one of [F1] to [F6] or the gene encoding the protein designated by any one of [f1] to [f6] is functionally linked to a promoter of a gene encoding HEV 2.1. As a result, the amount of cis isoprenoid biosynthesized in the laticifers can be enhanced, and therefore polyisoprenoid production can be improved in the plant. Further, it is also possible to increase the weight average molecular weight of polyisoprenoid to be produced. Thus, another aspect of the present invention is a method of increasing the weight average molecular weight of polyisoprenoid to be produced in a plant by introducing into the plant a vector comprising a base sequence in which a gene encoding cis-prenyltransferase is functionally linked to a promoter of a gene encoding HEV 2.1.

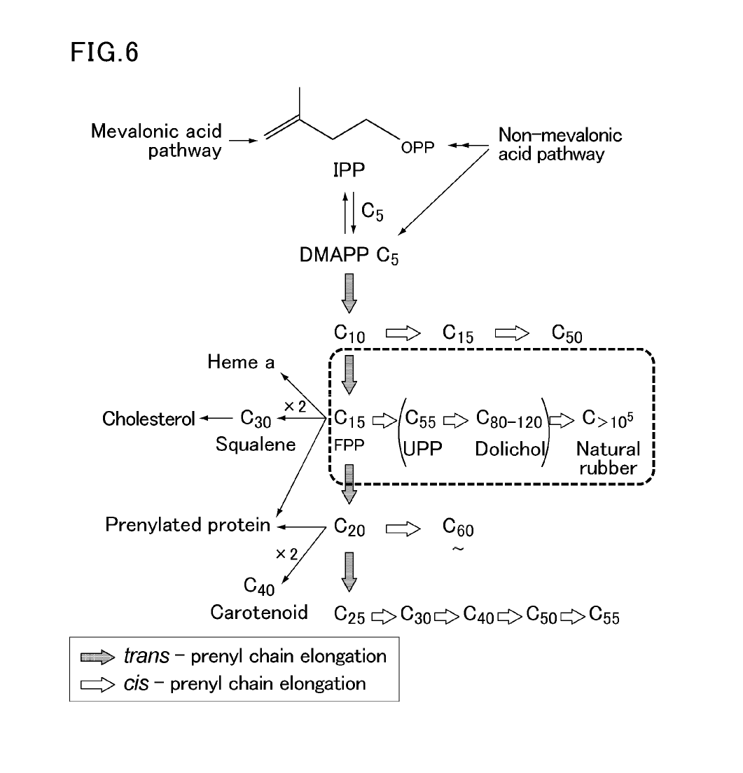

In a polyisoprenoid-producing plant, a polyisoprenoid is biosynthesized via the polyisoprenoid biosynthesis pathway as shown in FIG. 6, in which Small Rubber Particle Protein is considered to be involved in the catalytic reaction of the enzyme catalyzing the reaction enclosed by the dotted frame in FIG. 6. Accordingly, the expression of Small Rubber Particle Protein is increased specifically in laticifers by introducing into a plant a base sequence in which the gene designated by any one of [G1] to [G3] or the gene encoding the protein designated by any one of [g1] to [g3] is functionally linked to a promoter of a gene encoding HEV 2.1. As a result, the amount of cis isoprenoid biosynthesized in the laticifers can be enhanced, and therefore polyisoprenoid production can be improved in the plant. Further, it is also possible to increase the weight average molecular weight of polyisoprenoid to be produced. Thus, another aspect of the present invention is a method of increasing the weight average molecular weight of polyisoprenoid to be produced in a plant by introducing into the plant a vector comprising a base sequence in which a gene encoding Small Rubber Particle Protein is functionally linked to a promoter of a gene encoding HEV 2.1.

Rubber Elongation Factor is also considered to be involved in the catalytic reaction of the enzyme catalyzing the reaction enclosed by the dotted frame in FIG. 6. Accordingly, the expression of Rubber Elongation Factor is increased specifically in laticifers by introducing into a plant a base sequence in which the gene designated by any one of [H1] to [H3] or the gene encoding the protein designated by any one of [h1] to [h3] is functionally linked to a promoter of a gene encoding HEV 2.1. As a result, the amount of cis isoprenoid biosynthesized in the laticifers can be enhanced, and therefore polyisoprenoid production can be improved in the plant. Further, it is also possible to increase the weight average molecular weight of polyisoprenoid to be produced. Thus, another aspect of the present invention is a method of increasing the weight average molecular weight of the polyisoprenoid to be produced in a plant by introducing into the plant a vector comprising a base sequence in which a gene encoding Rubber Elongation Factor is functionally linked to a promoter of a gene encoding HEV 2.1.

In the present invention, though polyisoprenoid production in a plant is improved by introducing into the plant at least one of the genes encoding the seven kinds of proteins, polyisoprenoid production in a plant is further improved by introducing into the plant two or more of the genes encoding the seven kinds of proteins, and the effect of the invention is most significantly achieved by introducing all the genes of the seven kinds into the plant.

### EXAMPLES

The present invention will specifically be described with reference to Examples, but the invention is not limited to the Examples.

### (Preparation of Base Sequence of Promoter of Gene Encoding HEV2.1)

Cloning of a DNA fragment containing a gene encoding Hevein 2.1 (HEV2.1) derived from leaves of para rubber tree and its promoter was carried out by the following procedures. First, a genomic DNA was extracted from leaves of para rubber tree. The extraction was carried out by the CTAB method. A gene including the promoter region of the gene encoding HEV2.1 was amplified by TAIL-PCR using random primers shown as Primers 1 to 2 below and primers corresponding to the gene encoding HEV2.1.

(Primer 1: SEQ ID NO: 20)
5′-CTCAAGGCTACCTTATTGGG-3′
(Primer 2: SEQ ID NO: 21)
5′-CTCAGCAATTGCAACACCTG-3′

As a result of investigation of the base sequence of a DNA fragment obtained using the above-described primers, it was confirmed that the promoter sequence of the gene encoding HEV2.1 was obtained. The base sequence of the promoter sequence of the gene encoding HEV2.1 is shown as SEQ ID NO: 1.

(Amplification of Promoter Sequence for Introduction into Vector)

Primers 3 and 4 shown below to each of which a restriction enzyme site had been added were designed to be able to be incorporated into a vector for plants, and the above-identified promoter sequence −1 to −1680 bp of the gene encoding HEV2.1 was amplified by PCR.

(Primer 3: SEQ ID NO: 22)
5′-GGTACCGCTACCTTATTGGGAACTACC-3′
(Primer 4: SEQ ID NO: 23)
5′-AGATCTAACTOTTCCCATTTCTTCCC-3′

### (Identification of Amino Acid Sequences and Base Sequences of Target Proteins of Enzymes Involved in Polyisoprenoid Biosynthesis Pathway in Para Rubber Tree)

Total RNA was extracted from leaves of para rubber tree, and a cDNA was synthesized by reverse transcription reaction using oligo-dT primers.

Next, based on the DNA database of para rubber tree, Primers 5 to 22, 41, 42, 45, and 46 below were designed and the whole length DNA fragments of target proteins were amplified by RT-PCR to identify the base sequences of the DNA fragments and the whole length amino acid sequences of the target proteins. The base sequences of the genes encoding the target proteins are shown as SEQ ID NOs: 2, 4, 6 to 9, 14, 16, 18, 60, and 66, and the amino acid sequences of the target proteins are shown as SEQ ID NOs: 3, 5, 10 to 13, 15, 17, 19, 61, and 67.

The primers used when the target protein was farnesyl diphosphate synthase are shown below.

(Primer 5: SEQ ID NO: 24)
5′-GAATCCATGGCGGATCTGAAG-3′
(Primer 6: SEQ ID NO: 25)
5′-GTCCATGTATCTGGATACCC-3′

The primers used when the target protein was geranylgeranyl diphosphate synthase are shown below.

(Primer 7: SEQ ID NO: 26)
5′-CAAGATGAGTTCAGTGAATTTGGG-3′
(Primer 8: SEQ ID NO: 27)
5′-TGCATTAGTTTTGCCTGTGAGC-3′

The primers used when the target protein was 3-hydroxy-3-methylglutaryl CoA reductase 1 are shown below.

(Primer 9: SEQ ID NO: 28)
5′-ATTTTTACATGGACACCACCG-3′
(Primer 10: SEQ ID NO: 29)
5′-ACCAGATTCCCACTAAGATGC-3′

The primers used when the target protein was 3-hydroxy-3-methylglutaryl CoA reductase 3 are shown below.

(Primer 11: SEQ ID NO: 30)
5′-TCCATATATGGACGAGGTTCG-3′
(Primer 12: SEQ ID NO: 31)
5′-GCAGCTGTGTTACCCTTCAG-3′

The primers used when the target protein was 3-hydroxy-3-methylglutaryl CoA reductase 4 are shown below.

(Primer 13: SEQ ID NO: 32)
5′-CAGTCGCTCCAAAATGGATGTGC-3′
(Primer 14: SEQ ID NO: 33)
5′-GATTTTCTTAGGAAGAAGGCTTGG-3′

The primers used when the target protein was 3-hydroxy-3-methylglutaryl CoA reductase 5 are shown below.

(Primer 15: SEQ ID NO: 34)
5′-CTAGCTGGTCTATAATGGATGCC-3′
(Primer 16: SEQ ID NO: 35)
5′-GAATCAATTTACCTCAATAGAAGGC-3′

The primers used when the target protein was isopentenyl diphosphate isomerase are shown below.

(Primer 17: SEQ ID NO: 36)
5′-TTCCACCATGGGTGAGGCTCC-3′
(Primer 18: SEQ ID NO: 37)
5′-TCTCAACTCAACTTGTGAATCG-3′

The primers used when the target protein was cis-prenyltransferase 1 are shown below.

(Primer 19: SEQ ID NO: 38)
5′-ATGGAATTATACAACGGTGAGAGG-3′
(Primer 20: SEQ ID NO: 39)
5′-TTTTAAGTATTCCTTATGTTTCTCC-3′

The primers used when the target protein was cis-prenyltransferase 2 are shown below.

(Primer 45: SEQ ID NO: 68)
5′-ATGGAATTATACAACGGTGAGAGG-3′
(Primer 46: SEQ ID NO: 69)
5′-TTTTAAGTATTCCTTATGTTTCTCC-3′

The primers used when the target protein was Small Rubber Particle Protein are shown below.

(Primer 21: SEQ ID NO: 40)
5′-TATGGCTGAAGAGGTGGAGG-3′
(Primer 22: SEQ ID NO: 41)
5′-TGATGCCTCATCTCCAAACACC-3′

The primers used when the target protein was Rubber Elongation Factor are shown below.

(Primer 41: SEQ ID NO:. 62)
5′-ATGGCTGAAGACGAAGACAACC-3′
(Primer 42: SEQ ID NO: 63)
5′-ATTCTCTCCATAAAACACCTTAGC-3′

(Amplification of Base Sequences of Genes Encoding Target Proteins for Introduction into Vector)

Primers 23 to 40, 43, 44, 47, and 48 to each of which a restriction enzyme site had been added were designed to be able to be incorporated into a vector for plants, and the whole length genes encoding the target proteins were amplified by RT-PCR.

The primers used when the target protein was farnesyl diphosphate synthase are shown below.

(Primer 23: SEQ ID NO: 42)
5′-CTCGAGAACAATGGCGGATCTGAAGTCAAC-3′
(Primer 24: SEQ ID NO: 43)
5′-GGTACCTGTTTCTGTCTCTTGTAAATTTTGGC-3′

The primers used when the target protein was geranylgeranyl diphosphate synthase are shown below.

(Primer 25: SEQ ID NO: 44)
5′-CTCGAGAACAATGAGTTCAGTGAATTIGGG-3′
(Primer 26: SEQ ID NO: 45)
5′-GGATCCTTGTTTTGCCTGTGAGCGATGTAATTAGC-3′

The primers used when the target protein was 3-hydroxy-3-methylglutaryl CoA reductase 1 are shown below.

(Primer 27: SEQ ID NO: 46)
5′-CTCGAGACAAATGGACACCACCGGCCGGCTCC-3′
(Primer 28: SEQ ID NO: 47)
5′-GGTACCACAGATGCAGCTTTAGACATATCTTTGC-3′

The primers used when the target protein was 3-hydroxy-3-methylglutaryl CoA reductase 3 are shown below.

(Primer 29: SEQ ID NO: 48)
5′-CTCGAGACAAATGGACGAGGTTCGCCOGCGACC-3′
(Primer 30: SEQ ID NO: 49)
5′-GGTACCACGAAAGTTATTTTGGATACATCTTTTGC-3′

The primers used when the target protein was 3-hydroxy-3-methylglutaryl CoA reductase 4 are shown below.

(Primer 31: SEQ ID NO: 50)
5′-AAGCTTACAAATGGATGTGCGCCGGCGACC-3′
(Primer 32: SEQ ID NO: 51)
5′-GGTACCACGGAAGAAGGCTTGGAAACAGC-3′

The primers used when the target protein was 3-hydroxy-3-methylglutaryl CoA reductase 5 are shown below.

(Primer 33: SEQ ID NO: 52)
5′-AAGCTTACAAATGGATGCCCGCCGGCGACC-3′
(Primer 34: SEQ ID NO: 53)
5′-GGTACCACATTTACCTCAATAGAAGGCATTGTC-3′

The primers used when the target protein was isopentenyl diphosphate isomerase are shown below.

(Primer 35: SEQ ID NO: 54)
5′-CTCGAGAACAATGGGTGAGGCTCCAGATGTCG-3′
(Primer 36: SEQ ID NO: 55)
5′-GGTACCTGACTCAACTTGTGAATCGTTTTCATGTC-3′

The primers used when the target protein was cis-prenyltransferase 1 are shown below.

(Primer 37: SEQ ID NO: 56)
5′-CTCGAGCCAACAATGGAATTATACAACG-3′
(Primer 38: SEQ ID NO: 57)
5′-GGATCCTCTTTTAAGTATTCCTTATGTTTCTCC-3′

The primers used when the target protein was cis-prenyltransferase 2 are shown below.

(Primer 47: SEQ ID NO: 70)
5′-CTCGAGCCAACAATGGAATTATACAACG-3′
(Primer 48: SEQ ID NO: 71)
5′-GGATCCTCTTTTAAGTATTCCTTATGTTTCTCC-3′

The primers used when the target protein was Small Rubber Particle Protein are shown below.

(Primer 39: SEQ ID NO: 58)
5′-CTCGAGAACAATGGCTGAAGAGGTGGAGG-3′
(Primer 40: SEQ ID NO: 59)
5′-GGATCCTGTGATGCCTCATCTCCAAACACC-3′

The primers used when the target protein was Rubber Elongation Factor are shown below.

(Primer 43: SEQ ID NO: 64)
5′-CTCGAGAACAATGGCTGAAGACGAAGACAACC-3′
(Primer 44: SEQ ID NO: 65)
5′-GGATCCAAATTCTCTCCATAAAACACCTTAGC-3′

### (Construction of Expression Vectors)

A pH35GS vinery vector (Inplanta Innovations Inc.) was digested by restriction enzymes Kpn I and Bgl II, and the promoter sequence of the gene encoding HEV2.1 amplified in (Amplification of Promoter Sequence for Introduction into Vector) was treated with the restriction enzymes in the same manner and they were ligated using Ligation high (Toyobo Co., Ltd.).

Also, each of the genes (base sequences) encoding the target proteins amplified in (Amplification of Base Sequences of Genes Encoding Target Proteins for Introduction into Vector) was digested by restriction enzymes, and a Gateway sGFP entry clone vector (Evorogen) was treated with the restriction enzymes in the same manner and they were ligated using Ligation high (Toyobo Co., Ltd.).

The prepared binary vector and entry clone vector were reacted using LR Clonase II enzyme mix (Invitrogen) to insert the base sequence of the GFP (green fluorescent protein) gene and the gene sequence encoding the target protein on the entry clone into pH35GS. In this manner, expression vectors in each of which each of the gene sequences encoding the target proteins and the base sequence of the GFP gene were functionally linked to the promoter sequence (HbHEV2.1 pro) of the gene encoding HEV2.1 were constructed. The pH35GS vector had a hygromycin-resistant gene (HPT).

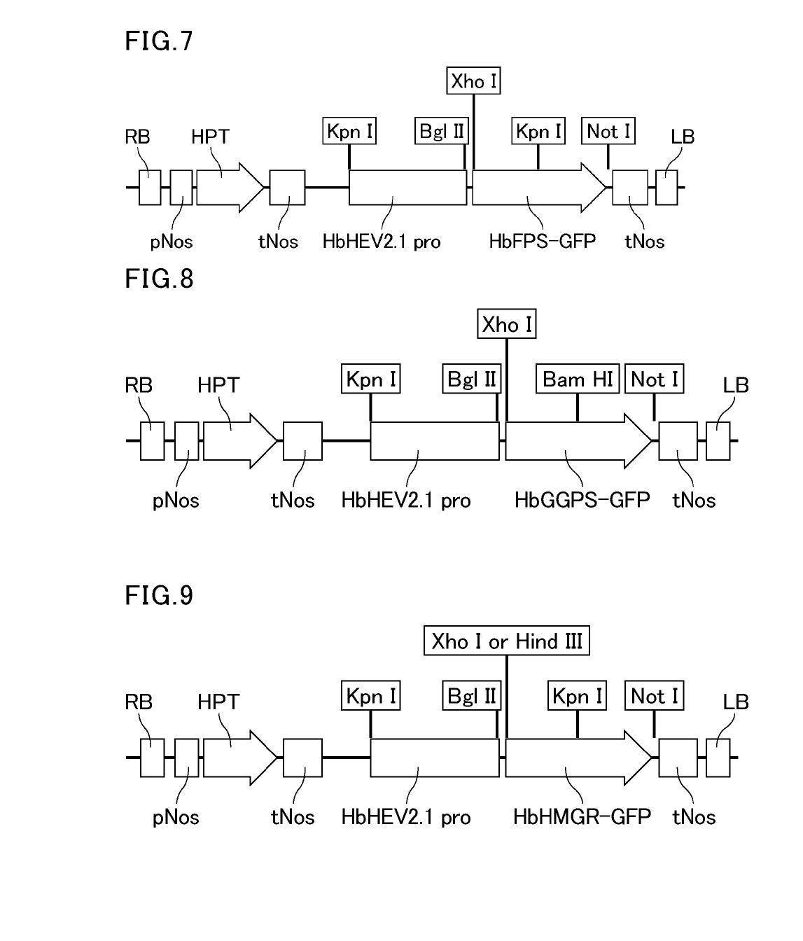

When the target protein was farnesyl diphosphate synthase, the gene encoding farnesyl diphosphate synthase and the Gateway sGFP entry clone vector were digested by restriction enzymes XhoI and KpnI. The thus-constructed expression vector is shown in FIG. 7. In FIG. 7, HbFPS represents the base sequence of the gene encoding farnesyl diphosphate synthase.

When the target protein was geranylgeranyl diphosphate synthase, the gene encoding geranylgeranyl diphosphate synthase and the Gateway sGFP entry clone vector were digested by restriction enzymes XhoI and BamHI. The thus-constructed expression vector is shown in FIG. 8, In FIG. 8, HbGGPS represents the base sequence of the gene encoding geranylgeranyl diphosphate synthase.

When the target protein was 3-hydroxy-3-methylglutaryl CoA reductase 1, 3, 4, or 5, the gene encoding 3-hydroxy-3-methylglutaryl CoA reductase 1, 3, 4, or 5 and the Gateway sGFP entry clone vector were digested by restriction enzymes XhoI and KpnI, or HindIII and KpnI. The thus-constructed expression vector is shown in FIG. 9. In FIG. 9, HbHMGR represents the base sequence of the gene encoding 3-hydroxy-3-methylglutaryl CoA reductase.

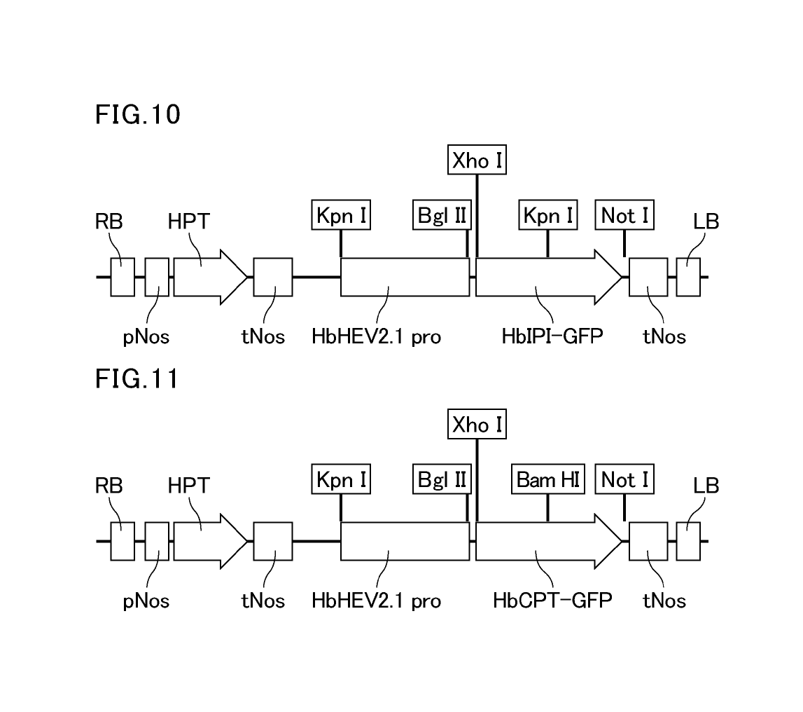

When the target protein was isopentenyl diphosphate isomerase, the gene encoding isopentenyl diphosphate isomerase and the Gateway sGFP entry clone vector were digested by restriction enzymes XhoI and KpnI. The thus-constructed expression vector is shown in FIG. 10. In FIG. 10, HbIPI represents the base sequence of the gene encoding isopentenyl diphosphate isomerase.

When the target protein was cis-prenyltransferase 1 or 2, the gene encoding cis-prenyltransferase 1 or 2 and the Gateway sGFP entry clone vector were digested by restriction enzymes XhoI and BamHI. The thus-constructed expression vector is shown in FIG. 11. In FIG. 11, HbCPT represents the base sequence of the gene encoding cis-prenyltransferase.

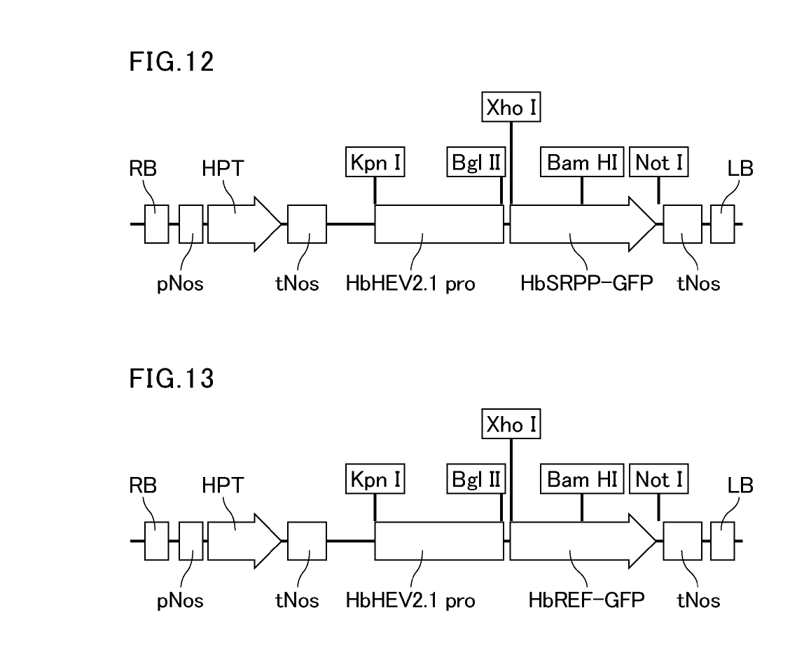

When the target protein was Small Rubber Particle Protein, the gene encoding Small Rubber Particle Protein and the Gateway sGFP entry clone vector were digested by restriction enzymes XhoI and BamHI. The thus-constructed expression vector is shown in FIG. 12. In FIG. 12, HbSRPP represents the base sequence of the gene encoding Small Rubber Particle Protein.

When the target protein was Rubber Elongation Factor, the gene encoding Rubber Elongation Factor and the Gateway sGFP entry clone vector were digested by restriction enzymes XhoI and BamHI. The thus-constructed expression vector is shown in FIG. 13. In FIG. 13, HbREF represents the base sequence of the gene encoding Rubber Elongation Factor.

### (Preparation of Expression Vector-Introduced Agrobacterium)

Each of the expression vectors constructed in (Construction of Expression Vectors) was infected with Agrobacterium to prepare an expression vector-introduced Agrobacterium. Specifically, the expression vectors were each introduced into Agrobacterium by electroporation and subjected to shake culture in YEB medium at 28° C. for 24 hours. After that, the culture liquid was centrifuged to recover the Agrobacterium, which was then suspended into a suspension (MS medium) at a concentration corresponding to O. D. 600=0.6.

### Examples 1 to 11

The chemicals used in Examples are listed below:

NAA: naphthaleneacetic acid;
2,4-D: 2,4-dichlorophenoxyacetic acid;
IBA: indolebutyric acid;
BA: benzyladenine;
KI: kinetin; and
para rubber tree: obtained from the Arboricultural Research Institute of the University of Tokyo Forests, Graduate School of Agricultural and Life Sciences, the University of Tokyo.

### (Callus Induction (Induction Step))

Leaves were collected from the para rubber tree. Then, after the surface of the collected leaves was washed in running water and then with 70% ethanol, they were sterilized with a sodium hypochlorite solution diluted to about 5 to 10% and washed again in running water.

Next, a tissue of the sterilized leaves was inserted into an induction medium (solid medium) and cultured (induction step). The induction medium was prepared by adding to MS medium (disclosed on pages 20 to 36 of Shokubutsu Saibo Kogaku Nyumon (Introduction to Plant Cell Engineering), Japan Scientific Societies Press) 2,4-dichlorophenoxyacetic acid (2,4-D), kinetin (KI), and sucrose at concentrations of 2.0 mg/l, 1.0 mg/l, and 3 mass %, respectively, adjusting the pH of the medium to 5.7 to 5.8, adding thereto gellan gum at a concentration of 0.2 mass %, sterilizing the medium in an autoclave (121° C., 20 minutes), and cooling it in a clean bench.

A tissue slice of the para rubber tree was inserted into an induction medium (solid medium) and cultured at a culture temperature of 25° C. in a dark place (0 to 0.1 lx) for 8 weeks to induce a callus (undifferentiated cells) from the tissue slice of the para rubber tree.

### (Infection Step and Coculture Step)

After the induced callus was contacted with the suspension (MS medium) containing the expression vector-introduced Agrobacterium prepared in (Preparation of Expression Vector-Introduced Agrobacterium) for 30 minutes, the callus was cultured in MS medium under dark place conditions at 28° C. for 3 days.

Here, the Agrobacterium into which the expression vector shown in FIG. 7 had been introduced was used in Example 1; the Agrobacterium into which the expression vector shown in FIG. 8 had been introduced was used in Example 2; the Agrobacterium into which the expression vector with HMGR1 shown in FIG. 9 had been introduced was used in Example 3; the Agrobacterium into which the expression vector shown in FIG. 10 had been introduced was used in Example 4; the Agrobacterium into which the expression vector with CPT1 shown in FIG. 11 had been introduced was used in Example 5; the Agrobacterium into which the expression vector shown in FIG. 12 had been introduced was used in Example 6; the Agrobacterium into which the expression vector shown in FIG. 13 had been introduced was used in Example 7; the Agrobacterium into which the expression vector with HMGR3 shown in FIG. 9 had been introduced was used in Example 8; the Agrobacterium into which the expression vector with HMGR4 shown in FIG. 9 had been introduced was used in Example 9; the Agrobacterium into which the expression vector with HMGR5 shown in FIG. 9 had been introduced was used in Example 10; and the Agrobacterium into which the expression vector with CPT2 shown in FIG. 11 had been introduced was used in Example 11.

### (Selective Culture Step)

Next, after the callus was sterilized with carbenicillin, it was transferred to a hygromycin-resistant MS selective medium and cultured at 28° C. with a 16 hour daylength for 2 months to prepare a transformant.

### (Formation of Adventitious Embryo and Shoot (Regeneration-Inducing Step))

Next, an adventitious embryo and a shoot were formed from the transformant (callus survived through the selective culture step) in MS medium (regeneration-inducing step). Specifically, the transformant was cultured in the medium at a culture temperature of 25° C. in 12 hours light (10000 lx) per 24 hour day for 3 to 6 months. During the culture, media were replaced every one month. As a result, it was observed that an adventitious embryo was formed and then a shoot was also formed.

### (Shoot Elongation (Elongation Step))

Next, for shoot elongation, the formed shoot was subcultured in a medium having the same composition as that of the regeneration-inducing medium. Specifically, the shoot was cultured at a culture temperature of 25° C. in 12 hours light (10000 lx) per 24 hour day for 8 weeks. As a result of the culture, good shoot elongation was observed.

### (Rooting (Rooting Step))

Next, for rooting, the shoot grown to about 3 cm was transplanted in ½ MS medium. The shoot was cultured at a culture temperature of 25° C. in 12 hours light (10000 lx) per 24 hour day for 8 weeks. As a result of the culture, good rooting was observed, whereby a transgenic plant was obtained.

### (Calculation of Polyisoprenoid Production and Measurement of Weight Average Molecular Weight of Polyisoprenoid)

A volume of 200 μl of latex exuded by cutting the stem of each of the transgenic plants obtained in Examples 1 to 7 was collected, and 100 μl out of the collected latex was extracted with 99% ethanol to remove unwanted metabolites. After that, it was further extracted with 99% toluene to purify and recover a polyisoprenoid. Then, the amount of the produced polyisoprenoid (recovered toluene extract) (the amount of the polyisoprenoid accumulated in the transgenic plant (intracellular accumulation of polyisoprenoid), i.e., polyisoprenoid production) was calculated.

The polyisoprenoid accumulation was calculated as a ratio (mass %) of the amount of polyisoprenoid purified and recovered from the transgenic plant to the amount of polyisoprenoid accumulated in the wild type (non-recombinant form; also referred to as “control”) after the recovered toluene extract was dried. The results are shown in Table 1.

The weight average molecular weight (Mw) of polyisoprenoid was measured by gel permeation chromatography (GPC) under the following conditions (1) to (7). The results are shown in Table 1.

### (1) Apparatus: HLC-8020 (Tosoh Corporation)

(2) Separation column: GMH-XL (Tosoh Corporation)
(3) Measurement temperature: 40° C.

### (4) Carrier: Tetrahydrofuran

(5) Flow rate: 0.6 ml/min.
(6) Detector: Differential refractometer, UV
(7) Molecular weight standards: Polystyrene standards

### Comparative Examples 1 to 7

Transgenic plants were obtained in the same manner as in Examples 1 to 7, except for using a cauliflower mosaic virus 35S promoter in place of the promoter of the gene encoding HEV2.1, i.e. except for not introducing the promoter sequence of the gene encoding HEV2.1 amplified in (Amplification of Promoter Sequence for Introduction into Vector). Then, polyisoprenoid accumulations were calculated and weight average molecular weights of polyisoprenoids were measured. The results are shown in Table 1.

TABLE 1

| | | | Comparative | | Comparative | | Comparative | |
|---|---|---|---|---|---|---|---|---|
| | Control | Example 1 | Example 1 | Example 2 | Example 2 | Example 3 | Example 3 | Example 4 |
| Promoter | — | HEV2.1 | 35S | HEV2.1 | 35S | HEV2.1 | 35S | HEV2.1 |
| | | pro | | pro | | pro | | pro |
| Protein encoded by gene | — | FPS | FPS | GGPS | GGPS | HMGR1 | HMGR1 | IPI |
| introduced into transgenic | | | | | | | | |
| plant | | | | | | | | |
| Polyisoprenoid | 100 | 105 | 101 | 102 | 100 | 114 | 102 | 106 |
| accumulation (% by mass) | | | | | | | | |
| Weight average molecular | 8.8 × 10⁵ | 9.0 × 10⁵ | 8.8 × 10⁵ | 9.1 × 10⁵ | 8.8 × 10⁵ | 1.2 × 10⁶ | 8.9 × 10⁵ | 8.8 × 10⁵ |
| weight (Mw) of | | | | | | | | |
| polyisoprenoid | | | | | | | | |

| | Comparative | | Comparative | | Comparative | | Comparative |
|---|---|---|---|---|---|---|---|
| | Example 4 | Example 5 | Example 5 | Example 6 | Example 6 | Example 7 | Example 7 |
| Promoter | 35S | HEV2.1 | 35S | HEV2.1 | 35S | HEV2.1 | 35S |
| | | pro | | pro | | pro | |
| Protein encoded by gene | IPI | CPT1 | CPT1 | SRPP | SRPP | REF | REF |
| introduced into transgenic | | | | | | | |
| plant | | | | | | | |
| Polyisoprenoid | 102 | 115 | 101 | 105 | 102 | 109 | 102 |
| accumulation (% by mass) | | | | | | | |
| Weight average molecular | 8.8 × 10⁵ | 1.4 × 10⁶ | 9.0 × 10⁵ | 9.1 × 10⁵ | 8.7 × 10⁵ | 9.1 × 10⁵ | 8.8 × 10⁵ |
| weight (Mw) of | | | | | | | |
| polyisoprenoid | | | | | | | |

The abbreviations in Table 1 stand for the following substances.

HEV2.1 pro: the promoter of the gene encoding HEV2.1 (Hevein 2.1)
35S: cauliflower mosaic virus 35S promoter
FPS: farnesyl diphosphate synthase
GGPS: geranylgeranyl diphosphate synthase
HMGR1: 3-hydroxy-3-methylglutaryl CoA reductase 1
IPI: isopentenyl diphosphate isomerase
CPT1: cis-prenyltransferase 1

### SRPP: Small Rubber Particle Protein

### REF: Rubber Elongation Factor

From the results shown in Table 1, it was demonstrated that the amount of polyisoprenoid accumulated in the plant was increased in the transgenic plants into which had been introduced the base sequence in which the gene encoding the protein involved in polyisoprenoid biosynthesis was functionally linked to the promoter of the gene encoding HEV 2.1 (Examples 1 to 7), as compared to the wild type (non-recombinant) and the transgenic plants obtained by using the cauliflower mosaic virus 35S promoter in place of the promoter of the gene encoding HEV2.1.

Further, it was also demonstrated that the weight average molecular weight of polyisoprenoid accumulated in the plant was increased particularly in the transgenic plants into which had been introduced the gene encoding farnesyl diphosphate synthase, geranylgeranyl diphosphate synthase, 3-hydroxy-3-methylglutaryl CoA reductase, cis-prenyltransferase, Small Rubber Particle Protein, or Rubber Elongation Factor as the protein involved in polyisoprenoid biosynthesis (Examples 1 to 3 and 5 to 7), as compared to the wild type (non-recombinant) and the transgenic plants obtained by using the cauliflower mosaic virus 35S promoter in place of the promoter of the gene encoding HEV2.1.

### Sequence Listing Free Text

SEQ ID NO: 1: base sequence of promoter of gene encoding Hevein 2.1 derived from para rubber tree
SEQ ID NO: 2: base sequence of gene encoding farnesyl diphosphate synthase derived from para rubber tree
SEQ ID NO: 3: amino acid sequence of farnesyl diphosphate synthase derived from para rubber tree
SEQ ID NO: 4: base sequence of gene encoding geranylgeranyl diphosphate synthase derived from para rubber tree
SEQ ID NO: 5: amino acid sequence of geranylgeranyl diphosphate synthase derived from para rubber tree
SEQ ID NO: 6: base sequence of gene encoding 3-hydroxy-3-methylglutaryl CoA reductase 1 derived from para rubber tree
SEQ ID NO: 7: base sequence of gene encoding 3-hydroxy-3-methylglutaryl CoA reductase 3 derived from para rubber tree
SEQ ID NO: 8: base sequence of gene encoding 3-hydroxy-3-methylglutaryl CoA reductase 4 derived from para rubber tree
SEQ ID NO: 9: base sequence of gene encoding 3-hydroxy-3-methylglutaryl CoA reductase 5 derived from para rubber tree
SEQ ID NO: 10: amino acid sequence of 3-hydroxy-3-methylglutaryl CoA reductase 1 derived from para rubber tree
SEQ ID NO: 11: amino acid sequence of 3-hydroxy-3-methylglutaryl CoA reductase 3 derived from para rubber tree
SEQ ID NO: 12: amino acid sequence of 3-hydroxy-3-methylglutaryl CoA reductase 4 derived from para rubber tree
SEQ ID NO: 13: amino acid sequence of 3-hydroxy-3-methylglutaryl CoA reductase 5 derived from para rubber tree
SEQ ID NO: 14: base sequence of gene encoding isopentenyl diphosphate isomerase derived from para rubber tree
SEQ ID NO: 15: amino acid sequence of isopentenyl diphosphate isomerase derived from para rubber tree
SEQ ID NO: 16: base sequence of gene encoding cis-prenyltransferase 1 derived from para rubber tree
SEQ ID NO: 17: amino acid sequence of cis-prenyltransferase 1 derived from para rubber tree
SEQ ID NO: 18: base sequence of gene encoding Small Rubber Particle Protein derived from para rubber tree
SEQ ID NO: 19: amino acid sequence of Small Rubber Particle Protein derived from para rubber tree

### SEQ ID NO: 20: Primer 1

### SEQ ID NO: 21: Primer 2

### SEQ ID NO: 22: Primer 3

### SEQ ID NO: 23: Primer 4

### SEQ ID NO: 24: Primer 5

### SEQ ID NO: 25: Primer 6

### SEQ ID NO: 26: Primer 7

### SEQ ID NO: 27: Primer 8

### SEQ ID NO: 28: Primer 9

### SEQ ID NO: 29: Primer 10

### SEQ ID NO: 30: Primer 11

### SEQ ID NO: 31: Primer 12

### SEQ ID NO: 32: Primer 13

### SEQ ID NO: 33: Primer 14

### SEQ ID NO: 34: Primer 15

### SEQ ID NO: 35: Primer 16

### SEQ ID NO: 36: Primer 17

### SEQ ID NO: 37: Primer 18

### SEQ ID NO: 38: Primer 19

### SEQ ID NO: 39: Primer 20

### SEQ ID NO: 40: Primer 21

### SEQ ID NO: 41: Primer 22

### SEQ ID NO: 42: Primer 23

### SEQ ID NO: 43: Primer 24

### SEQ ID NO: 44: Primer 25

### SEQ ID NO: 45: Primer 26

### SEQ ID NO: 46: Primer 27

### SEQ ID NO: 47: Primer 28

### SEQ ID NO: 48: Primer 29

### SEQ ID NO: 49: Primer 30

### SEQ ID NO: 50: Primer 31

### SEQ ID NO: 51: Primer 32

### SEQ ID NO: 52: Primer 33

### SEQ ID NO: 53: Primer 34

### SEQ ID NO: 54: Primer 35

### SEQ ID NO: 55: Primer 36

### SEQ ID NO: 56: Primer 37

### SEQ ID NO: 57: Primer 38

### SEQ ID NO: 58: Primer 39

### SEQ ID NO: 59: Primer 40

SEQ ID NO: 60: base sequence of gene encoding Rubber Elongation Factor derived from para rubber tree
SEQ ID NO: 61: amino acid sequence of Rubber Elongation Factor derived from para rubber tree

### SEQ ID NO: 62: Primer 41

### SEQ ID NO: 63: Primer 42

### SEQ ID NO: 64: Primer 43

### SEQ ID NO: 65: Primer 44

SEQ ID NO: 66: base sequence of gene encoding cis-prenyltransferase 2 derived from para rubber tree
SEQ ID NO: 67: amino acid sequence of cis-prenyltransferase 2 derived from para rubber tree

### SEQ ID NO: 68: Primer 45

### SEQ ID NO: 69: Primer 46

### SEQ ID NO: 70: Primer 47

### SEQ ID NO: 71: Primer 48

1. A vector, comprising:
a promoter of a gene encoding Hevein 2.1; and
a gene encoding a protein involved in polyisoprenoid biosynthesis, the gene being functionally linked to the promoter.

2. The vector according to claim 1, wherein the promoter of the gene encoding Hevein 2.1 comprises any one of the following DNAs:
[A1] a DNA comprising the base sequence of base numbers 1 to 1680 represented by SEQ ID NO: 1;
[A2] a DNA hybridizing to a DNA comprising a base sequence complementary to the base sequence of base numbers 1 to 1680 represented by SEQ ID NO: 1 under stringent conditions, and having a promoter activity for laticifer-specific gene expression; and
[A3] a DNA comprising a base sequence having 80% or more sequence identity with the base sequence of base numbers 1 to 1680 represented by SEQ ID NO: 1, and having a promoter activity for laticifer-specific gene expression.

3. The vector according to claim 1, wherein the gene encoding a protein involved in polyisoprenoid biosynthesis is at least one gene selected from the group consisting of a gene encoding farnesyl diphosphate synthase, a gene encoding geranylgeranyl diphosphate synthase, a gene encoding 3-hydroxy-3-methylglutaryl CoA reductase, a gene encoding isopentenyl diphosphate isomerase, a gene encoding cis-prenyltransferase, a gene encoding Small Rubber Particle Protein, and a gene encoding Rubber Elongation Factor.

4. The vector according to claim 3, wherein the gene encoding farnesyl diphosphate synthase comprises any one of the following DNAs:
[B1] a DNA comprising the base sequence of base numbers 1 to 1029 represented by SEQ ID NO: 2;
[B2] a DNA hybridizing to a DNA comprising abase sequence complementary to the base sequence of base numbers 1 to 1029 represented by SEQ ID NO: 2 under stringent conditions, and encoding a protein having an enzyme activity that catalyzes a reaction using isopentenyl diphosphate and dimethylallyl diphosphate as substrates or a reaction using isopentenyl diphosphate and geranyl diphosphate as substrates; and
[B3] a DNA comprising a base sequence having 80% or more sequence identity with the base sequence of base numbers 1 to 1029 represented by SEQ ID NO: 2, and encoding a protein having an enzyme activity that catalyzes a reaction using isopentenyl diphosphate and dimethylallyl diphosphate as substrates or a reaction using isopentenyl diphosphate and geranyl diphosphate as substrates.

5. The vector according to claim 3, wherein the gene encoding geranylgeranyl diphosphate synthase comprises any one of the following DNAs:
[C1] a DNA comprising the base sequence of base numbers 1 to 1113 represented by SEQ ID NO: 4;
[C2] a DNA hybridizing to a DNA comprising a base sequence complementary to the base sequence of base numbers 1 to 1113 represented by SEQ ID NO: 4 under stringent conditions, and encoding a protein having an enzyme activity that catalyzes a reaction using isopentenyl diphosphate and dimethylallyl diphosphate as substrates, a reaction using isopentenyl diphosphate and geranyl diphosphate as substrates, or a reaction using isopentenyl diphosphate and farnesyl diphosphate as substrates; and
[C3] a DNA comprising a base sequence having 80% or more sequence identity with the base sequence of base numbers 1 to 1113 represented by SEQ ID NO: 4, and encoding a protein having an enzyme activity that catalyzes a reaction using isopentenyl diphosphate and dimethylallyl diphosphate as substrates, a reaction using isopentenyl diphosphate and geranyl diphosphate as substrates, or a reaction using isopentenyl diphosphate and farnesyl diphosphate as substrates.

6. The vector according to claim 3, wherein the gene encoding 3-hydroxy-3-methylglutaryl CoA reductase comprises any one of the following DNAs:
[D1] a DNA comprising the base sequence of base numbers 1 to 1728 represented by SEQ ID NO: 6;
[D2] a DNA hybridizing to a DNA comprising a base sequence complementary to the base sequence of base numbers 1 to 1728 represented by SEQ ID NO: 6 under stringent conditions, and encoding a protein having an enzyme activity that catalyzes a reaction of reduction of 3-hydroxy-3-methylglutaryl CoA;
[D3] a DNA comprising a base sequence having 80% or more sequence identity with the base sequence of base numbers 1 to 1728 represented by SEQ ID NO: 6, and encoding a protein having an enzyme activity that catalyzes a reaction of reduction of 3-hydroxy-3-methylglutaryl CoA;
[D4] a DNA comprising the base sequence of base numbers 1 to 1761 represented by SEQ ID NO: 7;
[D5] a DNA hybridizing to a DNA comprising a base sequence complementary to the base sequence of base numbers 1 to 1761 represented by SEQ ID NO: 7 under stringent conditions, and encoding a protein having an enzyme activity that catalyzes a reaction of reduction of 3-hydroxy-3-methylglutaryl CoA;
[D6] a DNA comprising a base sequence having 80% or more sequence identity with the base sequence of base numbers 1 to 1761 represented by SEQ ID NO: 7, and encoding a protein having an enzyme activity that catalyzes a reaction of reduction of 3-hydroxy-3-methylglutaryl CoA;
[D7] a DNA comprising the base sequence of base numbers 1 to 1821 represented by SEQ ID NO: 8;
[D8] a DNA hybridizing to a DNA comprising a base sequence complementary to the base sequence of base numbers 1 to 1821 represented by SEQ ID NO: 8 under stringent conditions, and encoding a protein having an enzyme activity that catalyzes a reaction of reduction of 3-hydroxy-3-methylglutaryl CoA;
[D9] a DNA comprising a base sequence having 80% or more sequence identity with the base sequence of base numbers 1 to 1821 represented by SEQ ID NO: 8, and encoding a protein having an enzyme activity that catalyzes a reaction of reduction of 3-hydroxy-3-methylglutaryl CoA;
[D10] a DNA comprising the base sequence of base numbers 1 to 1581 represented by SEQ ID NO: 9;
[D11] a DNA hybridizing to a DNA comprising a base sequence complementary to the base sequence of base numbers 1 to 1581 represented by SEQ ID NO: 9 under stringent conditions, and encoding a protein having an enzyme activity that catalyzes a reaction of reduction of 3-hydroxy-3-methylglutaryl CoA; and
[D12] a DNA comprising a base sequence having 80% or more sequence identity with the base sequence of base numbers 1 to 1581 represented by SEQ ID NO: 9, and encoding a protein having an enzyme activity that catalyzes a reaction of reduction of 3-hydroxy-3-methylglutaryl CoA.

7. The vector according to claim 3, wherein the gene encoding isopentenyl diphosphate isomerase comprises any one of the following DNAs:
[E1] a DNA comprising the base sequence of base numbers 1 to 705 represented by SEQ ID NO: 14;
[E2] a DNA hybridizing to a DNA comprising a base sequence complementary to the base sequence of base numbers 1 to 705 represented by SEQ ID NO: 14 under stringent conditions, and encoding a protein having an enzyme activity that catalyzes a reaction of isomerization of isopentenyl diphosphate or dimethylallyl diphosphate; and
[E3] a DNA comprising a base sequence having 80% or more sequence identity with the base sequence of base numbers 1 to 705 represented by SEQ ID NO: 14, and encoding a protein having an enzyme activity that catalyzes a reaction of isomerization of isopentenyl diphosphate or dimethylallyl diphosphate.

8. The vector according to claim 3, wherein the gene encoding cis-prenyltransferase comprises any one of the following DNAs:
[F1] a DNA comprising the base sequence of base numbers 1 to 873 represented by SEQ ID NO: 16;
[F2] a DNA hybridizing to a DNA comprising a base sequence complementary to the base sequence of base numbers 1 to 873 represented by SEQ ID NO: 16 under stringent conditions, and encoding a protein having an enzyme activity that catalyzes a reaction of cis-chain elongation of isoprenoid compounds;
[F3] a DNA comprising a base sequence having 80% or more sequence identity with the base sequence of base numbers 1 to 873 represented by SEQ ID NO: 16, and encoding a protein having an enzyme activity that catalyzes a reaction of cis-chain elongation of isoprenoid compounds;
[F4] a DNA comprising the base sequence of base numbers 1 to 855 represented by SEQ ID NO: 66;
[F5] a DNA hybridizing to a DNA comprising a base sequence complementary to the base sequence of base numbers 1 to 855 represented by SEQ ID NO: 66 under stringent conditions, and encoding a protein having an enzyme activity that catalyzes a reaction of cis-chain elongation of isoprenoid compounds; and
[F6] a DNA comprising a base sequence having 80% or more sequence identity with the base sequence of base numbers 1 to 855 represented by SEQ ID NO: 66, and encoding a protein having an enzyme activity that catalyzes a reaction of cis-chain elongation of isoprenoid compounds.

9. The vector according to claim 3, wherein the gene encoding Small Rubber Particle Protein comprises any one of the following DNAs:
[G1] a DNA comprising the base sequence of base numbers 1 to 615 represented by SEQ ID NO: 18;
[G2] a DNA hybridizing to a DNA comprising a base sequence complementary to the base sequence of base numbers 1 to 615 represented by SEQ ID NO: 18 under stringent conditions, and encoding a rubber particle-associated protein which is associated with rubber particles in latex; and
[G3] a DNA comprising a base sequence having 80% or more sequence identity with the base sequence of base numbers 1 to 615 represented by SEQ ID NO: 18, and encoding a rubber particle-associated protein which is associated with rubber particles in latex.

10. The vector according to claim 3, wherein the gene encoding Rubber Elongation Factor comprises any one of the following DNAs:
[H1] a DNA comprising the base sequence of base numbers 1 to 417 represented by SEQ ID NO: 60;
[H2] a DNA hybridizing to a DNA comprising a base sequence complementary to the base sequence of base numbers 1 to 417 represented by SEQ ID NO: 60 under stringent conditions, and encoding a rubber particle-associated protein which is associated with rubber particles in latex; and
[H3] a DNA comprising a base sequence having 80% or more sequence identity with the base sequence of base numbers 1 to 417 represented by SEQ ID NO: 60, and encoding a rubber particle-associated protein which is associated with rubber particles in latex.

11. A transgenic plant into which the vector according to claim 1 has been introduced.

12. A method for improving polyisoprenoid production in a plant by introducing the vector according to claim 1 into the plant.


SEQUENCE LISTING (71 sequences)

SEQ ID NO: 1 - DNA, 1680 bp, Hevea brasiliensis
ggctaccttattgggaactaccaatttgtggattgtggtgattgaattaactaataagcaactgaatgttaatttccagaataagaaaacttgctgattgtaatctcaagttctagagtgaaaataaagataattatataaaatatatggaaattattatcctagaggaaattttatttttttttaaattaataaaatttttgtaattaaaaattttacgaaaaaaaatctaataaaataaatttatgtaaaattactttattttttataataaaataattacattatgtatgaaactaagtaatcatagaatatatatatatattatttagtttatgtgtcaaatataatagattaatattttctttattatttttcaaaataattttcatgtcaacccaattaaataaatatccaactaattttttttttaaatatttttatttcacagagaataatttgtatataaaaaataattttcataaaaatattttttattatttaattttaacattaattaatggtacgtgttcatattatatatgaataatatttttatattttaataaaattatcaaagttgagaaaatgatttgctcttttaagttctctcttaaaaaagaaagtcatttttcttaaaaataatttaatttctctttgactaaaatattttttgttaattatttttttaatactccaaacacaaaaaatgtgaaaaaaaaaatattttccacgacacaaacaaacagaattttagccaatcaattagcgcaattttcaactcccccgcctcctaaaggctggactggtgttgttcctggaggctgatatcctaagcaggtttctggatttgcactgattccatgatggttgaggcaagagggtatttctaatgagtttttatttagccctcttggttgttgcctgccactggaaatcaccatggaaacatatatgaagtcaaatgacaatttttattttttaaattttctgagagtgaggaaatgaataagaagaatttgttatttttctttaaagtcgtgttacttttacataatatattaagtcaaatttatcgactcagtgaaaataatttatattttataaataagaaaaatcttgttatataatttaatataaattttatatcttttttttttcaaggaaataaattttatatcttgatgataagatagagataagatcgagttaacccttgcgttaattggatgtttaaatgcttaatgcatggctaaggaaattaatgtctaaaataacagaaatgagaaaaataaatgaagggtgaaaaataaataaaacctggccctatgctctatattggggatggagtgggagccacctaatgtgtcagtgttcatcttcgaacaacgactcgattcaaagcacacccatgaagccgcttcacatcatccctttgaaactttccaccctaatcagctatcacacgatctactttccaatctcatcaacgctccaaatctcaccaccattcagtccactttcacttcctccttgtcctaatcatctttaatccatcggggtattatggtaattacatgatcaagtctctctgctataaataaagccaagtgagcttagctcatcatcacatcatttgcataccagaaaatcaagaattgggaagaaatgggaagagtt

SEQ ID NO: 2 - DNA, 1029 bp, Hevea brasiliensis
atggcggatctgaagtcaactttcttgaaggtctactctgtcctcaagcaggagctccttgaggatccggctttcgaatggacaccagattcccgtcaatgggttgagcggatgttggactacaatgtgcctggagggaagctgaatagggggctttctgtaattgacagctacaaattgttgaaagaaggacaggaattaacagaagaagagatctttcttgcaagtgctcttggttggtgtattgaatggcttcaagcctattttcttgtacttgatgacattatggatagctctcatacacgacgtggtcagccttgctggtttagggtgcccaaggttggtctgattgcagcaaatgatgggattttgcttcgcaatcacattcccaggattcttaaaaagcacttccgagggaaggcatactatgtagatcttctagatttgtttaatgaggtggagtttcaaacagcctcaggacagatgatagatctaattacaacacttgaaggagaaaaggatttatccaaatacactttgtcactccaccggagaattgttcagtacaaaactgcctactactcattttaccttcctgttgcttgtgcattgctcatagcgggtgagaatctggacaatcatattgttgtaaaagacattcttgttcagatgggaatctacttccaagtacaggatgattatttggattgctttggtgatcccgagacaattggtaagataggaacagatatagaagattttaagtgttcatggttggtcgtgaaggctttagaactttgcaatgaagaacaaaagaaagtgttatatgagcactatgggaaagctgacccagccagtgtagcaaaggtgaaggtcctttataatgagctgaagcttcagggggtatttacggagtatgagaatgaaagctataagaaactagtaacctctattgaagctcatcctagcaagccggtgcaagcagtgttgaagtcctttttggccaaaatttacaagagacagaaataa

SEQ ID NO: 3 - protein, 342 aa, Hevea brasiliensis
MADLKSTFLKVYSVLKQELLEDPAFEWTPDSRQWVERMLDYNVPGGKLNRGLSVIDSYKLLKEGQELTEEEIFLASALGWCIEWLQAYFLVLDDIMDSSHTRRGQPCWFRVPKVGLIAANDGILLRNHIPRILKKHFRGKAYYVDLLDLFNEVEFQTASGQMIDLITTLEGEKDLSKYTLSLHRRIVQYKTAYYSFYLPVACALLIAGENLDNHIVVKDILVQMGIYFQVQDDYLDCFGDPETIGKIGTDIEDFKCSWLVVKALELCNEEQKKVLYEHYGKADPASVAKVKVLYNELKLQGVFTEYENESYKKLVTSIEAHPSKPVQAVLKSFLAKIYKRQK

SEQ ID NO: 4 - DNA, 1113 bp, Hevea brasiliensis
atgagttcagtgaatttgggctcatgggttcacacctcctacgtcttaaaccaagctaccagatccagatccaaatccaaatccttctctctacctttcaatcctctaaaaagtttagcaatttcctttgcttatagaaaatcagagcgacccatttcatctgtctctgcgattattaccaaggaagaagaaactcttcaagaagagcagaataatccaccaccctcttttgatttcaaatcctacatgctccaaaaaggcaattccattaaccaagctttagaagctgccattccactccaagaacccgctaaaattcacgagtctatgcgttattccctcttggccggcggcaagagggtacgaccggccctctgcctcgctgcgtgtgagcttgttggtgggaatgactccatggcgatgcctgctgcatgcgctgtggaaatgattcatactatgtctcttatccatgatgacctcccttgcatggataacgacgatctccgccgtggcaagcccaccaatcacatcgtgtttggagaggacgtggcggttctcgccggtgacgcactcctagcatttgcttttgaacacatcgctgtttctactttaaatgtttcttctgctagaattgtccgggcagttggggaattagcgaaggcgatcggggcagaagggttagttgctggccaagtagttgatataaattctgagggctcatctgaggtggatttagagaagcttgaatttattcacatccacaagaccgctaagttgttggagggggctgtggtgctaggggctatattgggcggaggaaccgatgaggaagtggagaaattgaggaaatatgctagggatattgggttgttgttccaagttgttgacgatattcttgatgtgactaaatcatcccaagaattggggaaaactgcaggcaaggacttggtggcggacaaggttacatatcccaagcttttggggattgagaagtcgagggaatttgcagagaagctgaataaggaagctcaggagcagctggctggatttgatcctgaaaaggcagctccattgattgctttggctaattacatcgctcacaggcaaaactaa

SEQ ID NO: 5 - protein, 370 aa, Hevea brasiliensis
MSSVNLGSWVHTSYVLNQATRSRSKSKSFSLPFNPLKSLAISFAYRKSERPISSVSAIITKEEETLQEEQNNPPPSFDFKSYMLQKGNSINQALEAAIPLQEPAKIHESMRYSLLAGGKRVRPALCLAACELVGGNDSMAMPAACAVEMIHTMSLIHDDLPCMDNDDLRRGKPTNHIVFGEDVAVLAGDALLAFAFEHIAVSTLNVSSARIVRAVGELAKAIGAEGLVAGQVVDINSEGSSEVDLEKLEFIHIHKTAKLLEGAVVLGAILGGGTDEEVEKLRKYARDIGLLFQVVDDILDVTKSSQELGKTAGKDLVADKVTYPKLLGIEKSREFAEKLNKEAQEQLAGFDPEKAAPLIALANYIAHRQN

SEQ ID NO: 6 - DNA, 1728 bp, Hevea brasiliensis
atggacaccaccggccggctccaccaccgaaagcatgctacacccgttgaggaccgttctccgaccactccgaaagcgtcggacgcgcttccgcttcccctctacctgaccaacgcggttttcttcacgctgttcttctcggtggcgtattacctccttcaccggtggcgcgacaagatccgcaactccactccccttcatatcgttactctctctgaaattgttgctattgtctccctcattgcctctttcatttacctcctaggattcttcggtatcgattttgtgcagtcattcattgcacgcgcctcccatgacgtgtgggacctcgaagatacggatcccaactacctcatcgatgaagatcaccgtctcgttacttgccctcccgctaatatatctactaagactaccattattgccgcacctaccaaattgcctacctcggaacccttaattgcacccttagtctcggaggaagacgaaatgatcgtcaactccgtcgtggatgggaagataccctcctattctctggagtcgaagctcggggactgcaaacgagcggctgcgattcgacgcgaggctttgcagaggatgacaaggaggtcgctggaaggcttgccagtagaagggttcgattacgagtcgattttaggacaatgctgtgaaatgccagtgggatacgtgcagattccggtggggattgcggggccgttgttgctgaacgggcgggagtactctgttccaatggcgaccacggagggttgtttggtggcgagcactaatagagggtgtaaggcgatttacttgtcaggtggggccaccagcgtcttgttgaaggatggcatgacaagagcgcctgttgtaagattcgcgtcggcgactagagccgcggagttgaagttcttcttggaggatcctgacaattttgataccttggccgtagtttttaacaagtccagtagatttgcgaggctccaaggcattaaatgctcaattgctggtaagaatctttatataagattcagctacagcactggcgatgcaatggggatgaacatggtttctaaaggggttcaaaacgttcttgaatttcttcaaagtgatttttctgatatggatgtcattggaatctcaggaaatttttgttcggataagaagcctgctgctgtaaattggattgaaggacgtggcaaatcagttgtttgtgaggcaattatcaaggaagaggtggtgaagaaggtgttgaaaaccaatgtggcctccctagtggagcttaacatgctcaagaatcttgctggttctgctgttgctggtgctttgggtggatttaatgcccatgcaggcaacatcgtatctgcaatctttattgccactggccaggatccagcacagaatgttgagagttctcattgcattaccatgatggaagctgtcaatgatggaaaggatctccatatctctgtgaccatgccctccattgaggtgggtacagtcggaggtggaactcaacttgcatctcagtctgcttgtctcaatttgcttggggtgaagggtgcaaacaaagagtcgccaggatcaaactcaaggctccttgctgccatcgtagctggttcagttttggctggtgagctctccttgatgtctgccattgcagctgggcagcttgtcaagagtcacatgaagtacaacagatccagcaaagatatgtctaaagctgcatcttag

SEQ ID NO: 7 - DNA, 1761 bp, Hevea brasiliensis
atggacgaggttcgccggcgaccccccaagcatatcgtccggaaggaccacgacggcgaagttctcaactccttcagccatggccaccatcttcctcctctcaagccgtctgattattctctccctctctccctctacctcgctaatgctctcgttttctcgctcttcttctcagttgcttatttccttctccacagatggcgggaaaagatccgcaaatccactcctcttcacatagtcaccttccctgagattgctgctttgatttgcttggtagcttctgtcatctatcttcttggtttctttggcattggcttcgtccactccttttctagagcttccaccgattcctgggatgtcgaagaatacgatgatgataacattatcatcaaggaagatactcgtcctacgggggcttgcgctgcaccttcccttgactgctccctctctcttcccaccaaaattcatgcacccattgtctcgactacaaccaccagtactttatctgatgatgacgaacaaattatcaaatcagtagtctctggctccattccttcctattcccttgaatcaaagcttgggaactgtaagcgggctgctttaattcgccgggagacgctgcagaggatgtcggggagatccttggagggactgcccttagatgggttcgattacgagtcaatattggggcagtgctgcgagatggcaattgggtacgtgcagattccggtgggaattgctgggccattgttgctggatggaaaggaatatactgtgccaatggccacgaccgagggttgccttgtggccagtgcaaatagaggatgcaaggccatttacgcatcgggaggggctacctccgtgttgctgagggacggcatgacccgtgctcccgttgttaggtttcccaccgccaagagggccgctgacttgaagtttttcatggaggaccctgacaatttcgataccattgccgttgttttcaataagtcaagcagatttgcgaggctacagagtgtccaatgtgcaatagcaggaaaaaatttatacatgaggtttagctgcagcacaggtgatgccatggggatgaatatggtctccaaagcggtccaaaatgtcatcgattatctccagaatgattttcctgacatggatgtcatcggtctcactgggaacttctgtgcggacaagaaggcagcagcagtaaactggatagaagggcgtgggaagtctgttgtatgtgaagcaatcataaaggaagaggtggttaagaaggtattgaaaaccaacgtggccgccctggtggagcttaacatgattaaaaaccttactggctcagccgtagcaggttctcttggtgggtttaacgcccatgctagcaatatggtaactgcagtatacatagctacagggcaggatcctgctcaaaacgtggagagctctcactgcattaccatgatggaagctgttaacgatggcaaggaccttcacatctcagtgtccatgccttccattgagctgggcacagttggaggtggtactcaacttgcatctcaatcagcttgtctgaacctacttggggtaaagggtgcaagcaaagattcccctggttcaaactcaaggcttctggcaactattgtcgctggttctgttctggcaggggagctgtctcttatgtctgctattgcagctgggcaactcgttaatagccacatgaaatacaacagatctgcaaaagatgtatccaaaataactttctga

SEQ ID NO: 8 - DNA, 1821 bp, Hevea brasiliensis
atggatgtgcgccggcgacctacctctggaaaaaccatccactctgtgaaacctaaatcggtggaggatgagagtgcccagaagccctctgatgcattacctctccctctgtacctaatcaatgctctctgctttaccgttttcttttatgttgtttattttctgctcagccgctggcgtgagaagattcgcacatctacccctctacatgtcgttgctctctccgaaattgctgctattgttgctttcgttgcttccttcatttacctacttggcttctttggtattgactttgttcagtccctgatattacgccctcctacagatatgtgggctgtggatgatgatgaggaggagacggaggaagggattgtactcagggaagatacccggaaattgccatgtgggcaagctctcgattgctctctttctgcaccaccactgtcacgggcagttgtttcttctccgaaagcaatggaccctatcgtgcttccttctccgaagccgaaagtgttcgacgaaatcccatttccgacaaccaccaccatccctattttgggcgatgaagatgaggagatcattaaatctgttgtggctggaactatcccttcgtattccctcgagtctaaattgggggattgtaagcgtgctgctgcaatcaggcgcgaggccttgcagaggataactggcaagtctctctctgggttaccactggagggctttgattacgagtcaatcttggggcagtgttgcgagatgccagttgggtatgtccaaattcccgttggaattgctggacctttgttgctagacggcaaggaatactcggttcccatggctaccactgaagggtgcttggtagccagcaccaataggggttgcaaggccattcacttatctggtggagccacaagtgtcctgttgagagatgggatgaccagagcgccggtcgttaggtttgggacagcaaaaagagcggcccagttgaagttgtacttggaggatcctgccaattttgagaccctgtccacttcgtttaacaaatccagcagatttggcaggcttcaaagcatcaaatgcgctattgccggaaagaacctatacatgagattctgttgcagcactggtgacgctatgggtatgaacatggtctctaaaggtgtccagaatgtattgaatttcctccagaatgatttccctgacatggatgttattggcctttctggtaacttctgctcggataagaagcctgcagctgtgaactggattgaaggaaggggcaagtcagtggtgtgcgaggccataatcaagggagatgtggtgaagaaggtgttgaagacgaatgtggaagccttagtggagcttaacatgctcaagaacctcactggttcagccatggctggagctttaggagggttcaatgctcatgccagtaacatagtgactgcaatttacatagcaaccggccaagatccagcacagaacgtagagagttctaactgcatcaccatgatggaagctgttaatgatggacaggatcttcatgtctctgtgactatgccttctatcgaggttggtactgttggaggaggtactcagcttgcatctcaatcagcatgcctgaacctgcttggggtgaagggagcaagcaaagagacgccaggagctaactccagagttctagcctcaatagttgctggttctgttcttgctgctgagctatctctcatgtctgcaattgctgctggacaactagtgaacagccacatgaaatacaacagagccaacaaagaagctgctgtttccaagccttcttcctaa

SEQ ID NO: 9 - DNA, 1581 bp, Hevea brasiliensis
atggatgcccgccggcgacccacctccgggaatcccatccattcccgcaaagtaaaagcattggaggatgagaatacccagaagccctccgatgcactgcctctccatatctacctaaccaatgctctctgcttcaccgtcttcttttgggttgtttattttcttcttagccgctggcgtgagaagattcgcacctccgcccctctgcatgttgtcactctctctgaaattgctgctattgttgctttgtttgcttccttcatttaccttcttggcttctttggtattgattttgttcagtcccttatcttacgccctccaaccgatatgtgggctgtggatgatgaagaggaggagccggcggaacaaattctgctcaaggaagatgcccggaaatcgccatgtgggcaagctctcgattgctctcttactgcaccaccactatcacggccaattgtttcttcttcaaaagcagtgggcccaattgtgcttccttctccgaagccgaaagtggtcgaggaaatctcatttccagccatcactaccaccgccactttgggcgaggaagatgaggagatcatcaaatctgttgtggctggaactaccccttcgtattccctcgagtctaaattgggggattgcaagcgtgctgctgcaatcaggcgggaggcgttgcagaggataactggcaaatcgctttctgggttgcctctggagggctttgattatgagtcaatattgggacagtgctgcgagatgcccgttgggtatgtccagattcccgttggcattgctggacctttgttgctagacggtaaggaagtttcggttcccatggctaccactgaagggtgcttggtagccagtaccaataggggctgcaaggccattcacatatctggtggagccacaagcgtcgtgttgagggatgggatgaccagggcacctgttgttaggttcgggacggcaaaaagagcagcccaattgaagttttacttggaggatagtgccaattttgagactttgtctactgtgtttaacaaatccagcagatttggcaggcttcaaagtattaggtgcgctattgctggaaagaacctgtacattagattctgttgcggcactggtgacgctatgggcatgaacatggtgtctaaaggtgtccagaatgtattggatttcctccagaatgatttccctgacatggatgttattggcgtctctggtaacttctgctcggataagaagcctgcagctgtgaactggattgaaggaaggggaaagtcagtagcgtgcgaagccataatcaagggtgatgtggtgaagaaggtgttgaagacgaatgtggaagccttggtggagcttaatatgctgaagaatctaactggttctgccctggctggagctctaggtgggttcaatgctcatgccagtaacatagtgactgctatctacatagcgacaggccaagatcctgctcagaacgttgagagttctcactgtatcaccatgatggaagctgttaatgatgggcaggatcttcacgtctctgtgacaatgccttctattgaggtaaattga

SEQ ID NO: 10 - protein, 575 aa, Hevea brasiliensis
MDTTGRLHHRKHATPVEDRSPTTPKASDALPLPLYLTNAVFFTLFFSVAYYLLHRWRDKIRNSTPLHIVTLSEIVAIVSLIASFIYLLGFFGIDFVQSFIARASHDVWDLEDTDPNYLIDEDHRLVTCPPANISTKTTIIAAPTKLPTSEPLIAPLVSEEDEMIVNSVVDGKIPSYSLESKLGDCKRAAAIRREALQRMTRRSLEGLPVEGFDYESILGQCCEMPVGYVQIPVGIAGPLLLNGREYSVPMATTEGCLVASTNRGCKAIYLSGGATSVLLKDGMTRAPVVRFASATRAAELKFFLEDPDNFDTLAVVFNKSSRFARLQGIKCSIAGKNLYIRFSYSTGDAMGMNMVSKGVQNVLEFLQSDFSDMDVIGISGNFCSDKKPAAVNWIEGRGKSVVCEAIIKEEVVKKVLKTNVASLVELNMLKNLAGSAVAGALGGFNAHAGNIVSAIFIATGQDPAQNVESSHCITMMEAVNDGKDLHISVTMPSIEVGTVGGGTQLASQSACLNLLGVKGANKESPGSNSRLLAAIVAGSVLAGELSLMSAIAAGQLVKSHMKYNRSSKDMSKAAS

SEQ ID NO: 11 - protein, 586 aa, Hevea brasiliensis
MDEVRRRPPKHIVRKDHDGEVLNSFSHGHHLPPLKPSDYSLPLSLYLANALVFSLFFSVAYFLLHRWREKIRKSTPLHIVTFPEIAALICLVASVIYLLGFFGIGFVHSFSRASTDSWDVEEYDDDNIIIKEDTRPTGACAAPSLDCSLSLPTKIHAPIVSTTTTSTLSDDDEQIIKSVVSGSIPSYSLESKLGNCKRAALIRRETLQRMSGRSLEGLPLDGFDYESILGQCCEMAIGYVQIPVGIAGPLLLDGKEYTVPMATTEGCLVASANRGCKAIYASGGATSVLLRDGMTRAPVVRFPTAKRAADLKFFMEDPDNFDTIAVVFNKSSRFARLQSVQCAIAGKNLYMRFSCSTGDAMGMNMVSKAVQNVIDYLQNDFPDMDVIGLTGNFCADKKAAAVNWIEGRGKSVVCEAIIKEEVVKKVLKTNVAALVELNMIKNLTGSAVAGSLGGFNAHASNMVTAVYIATGQDPAQNVESSHCITMMEAVNDGKDLHISVSMPSIELGTVGGGTQLASQSACLNLLGVKGASKDSPGSNSRLLATIVAGSVLAGELSLMSAIAAGQLVNSHMKYNRSAKDVSKITF

SEQ ID NO: 12 - protein, 606 aa, Hevea brasiliensis
MDVRRRPTSGKTIHSVKPKSVEDESAQKPSDALPLPLYLINALCFTVFFYVVYFLLSRWREKIRTSTPLHVVALSEIAAIVAFVASFIYLLGFFGIDFVQSLILRPPTDMWAVDDDEEETEEGIVLREDTRKLPCGQALDCSLSAPPLSRAVVSSPKAMDPIVLPSPKPKVFDEIPFPTTTTIPILGDEDEEIIKSVVAGTIPSYSLESKLGDCKRAAAIRREALQRITGKSLSGLPLEGFDYESILGQCCEMPVGYVQIPVGIAGPLLLDGKEYSVPMATTEGCLVASTNRGCKAIHLSGGATSVLLRDGMTRAPVVRFGTAKRAAQLKLYLEDPANFETLSTSFNKSSRFGRLQSIKCAIAGKNLYMRFCCSTGDAMGMNMVSKGVQNVLNFLQNDFPDMDVIGLSGNFCSDKKPAAVNWIEGRGKSVVCEAIIKGDVVKKVLKTNVEALVELNMLKNLTGSAMAGALGGFNAHASNIVTAIYIATGQDPAQNVESSNCITMMEAVNDGQDLHVSVTMPSIEVGTVGGGTQLASQSACLNLLGVKGASKETPGANSRVLASIVAGSVLAAELSLMSAIAAGQLVNSHMKYNRANKEAAVSKPSS

SEQ ID NO: 13 - protein, 526 aa, Hevea brasiliensis
MDARRRPTSGNPIHSRKVKALEDENTQKPSDALPLHIYLTNALCFTVFFWVVYFLLSRWREKIRTSAPLHVVTLSEIAAIVALFASFIYLLGFFGIDFVQSLILRPPTDMWAVDDEEEEPAEQILLKEDARKSPCGQALDCSLTAPPLSRPIVSSSKAVGPIVLPSPKPKVVEEISFPAITTTATLGEEDEEIIKSVVAGTTPSYSLESKLGDCKRAAAIRREALQRITGKSLSGLPLEGFDYESILGQCCEMPVGYVQIPVGIAGPLLLDGKEVSVPMATTEGCLVASTNRGCKAIHISGGATSVVLRDGMTRAPVVRFGTAKRAAQLKFYLEDSANFETLSTVFNKSSRFGRLQSIRCAIAGKNLYIRFCCGTGDAMGMNMVSKGVQNVLDFLQNDFPDMDVIGVSGNFCSDKKPAAVNWIEGRGKSVACEAIIKGDVVKKVLKTNVEALVELNMLKNLTGSALAGALGGFNAHASNIVTAIYIATGQDPAQNVESSHCITMMEAVNDGQDLHVSVTMPSIEVN

SEQ ID NO: 14 - DNA, 705 bp, Hevea brasiliensis
atgggtgaggctccagatgtcggcatggatgctgtccagaaacgcctcatgttcgacgatgaatgcattttagtagatgagaacgatggtgttgttggtcatgcttccaaatataattgtcatttgtgggaaaatattttgaaggggaacgcattacatagagcttttagcgtatttctcttcaactcaaaatatgagctactccttcagcaacgctctgggacaaaggtgacattcccgcttgtatggacaaacacttgctgtagtcatcctctgtaccgtgaatctgagcttattgatgaggatgctcttggtgtgagaaatgctgcacaaaggaagcttttcgatgagcttggtatccctgctgaagatgttccagttgatcagtttactccactaggacgtatactatataaggcgtcctccgatggaaagtggggagagcatgaacttgattatctgctctttatagtccgtgatgttaatgtaaatccaaaccctgatgaggtagctgatgtaaagtatgttaaccgggatcagttgaaggagctcttgaggaaggcggattctggcgaggaaggtataaatttgtcaccttggtttagactagttgtggacaacttcttgttgaaatggtgggaaaatgtcgaaaatgggacactcaaggaagcagttgacatgaaaacgattcacaagttgagttga

SEQ ID NO: 15 - protein, 234 aa, Hevea brasiliensis
MGEAPDVGMDAVQKRLMFDDECILVDENDGVVGHASKYNCHLWENILKGNALHRAFSVFLFNSKYELLLQQRSGTKVTFPLVWTNTCCSHPLYRESELIDEDALGVRNAAQRKLFDELGIPAEDVPVDQFTPLGRILYKASSDGKWGEHELDYLLFIVRDVNVNPNPDEVADVKYVNRDQLKELLRKADSGEEGINLSPWFRLVVDNFLLKWWENVENGTLKEAVDMKTIHKLS

SEQ ID NO: 16 - DNA, 873 bp, Hevea brasiliensis
atgaaattatacaccggtgagaggccaagtgtgttcagacttttagggaagtatatgagaaaagggttatatggcatcctaacccagggtcccatccctactcatcttgccttcatattggatggaaacaggaggtttgctaagaagcataaactgccagaaggaggtggtcataaggctggatttttagctcttctgaacgtgctaacttattgctatgagttaggagtgaaatatgcgactatctatgcctttagcatcgataattttcgaaggaaacctcatgaggttcagtacgtaatgaatctaatgctggagaagattgaagggatgatcatggaagaaagtatcatcaatgcatatgatatttgcgtacgttttgtgggtaacctgaagcttttaagtgagccagtcaagaccgcagcagataagattatgagggctactgccaacaattccaaatgtgtgcttctccttgctgtatgctatacttcaactgatgagatcgtgcatgctgttgaagaatcctctgaattgaactccaatgaagtttgtaacaatcaagaattggaggaggcaaatgcaactggaagcggtactgtgattcaaactgagaacatggagtcgtattctggaataaaacttgtagaccttgagaaaaacacctacataaatccttatcctgatgttctgattcgaacttctggggagacccgtctgagcaactacttactttggcagactactaattgcatactgtattctccttatgcactgtggccagagattggtcttcgacacgtggtgtggtcagtaattaacttccaacgtcattattcttacttggagaaacataaggaatacttaaaataa

SEQ ID NO: 17 - protein, 290 aa, Hevea brasiliensis
MKLYTGERPSVFRLLGKYMRKGLYGILTQGPIPTHLAFILDGNRRFAKKHKLPEGGGHKAGFLALLNVLTYCYELGVKYATIYAFSIDNFRRKPHEVQYVMNLMLEKIEGMIMEESIINAYDICVRFVGNLKLLSEPVKTAADKIMRATANNSKCVLLLAVCYTSTDEIVHAVEESSELNSNEVCNNQELEEANATGSGTVIQTENMESYSGIKLVDLEKNTYINPYPDVLIRTSGETRLSNYLLWQTTNCILYSPYALWPEIGLRHVVWSVINFQRHYSYLEKHKEYLK

SEQ ID NO: 18 - DNA, 615 bp, Hevea brasiliensis
atggctgaagaggtggaggaagagaggctaaagtatttggattttgtgcgagcggctggagtttatgctgtagattctttctcaactctctacctttatgccaaggacatatctggtccattaaaacctggtgtcgatactattgagaatgtggtgaagaccgtggttactcctgtttattatattccccttgaggctgtcaagtttgtagacaaaacggtggatgtatcggtcactagcctagatggcgttgttcccccagttatcaagcaggtgtctgcccaaacttactcggtagctcaagatgctccaagaattgttcttgatgtggcttcttcagttttcaacactggtgtgcaggaaggcgcaaaagctctgtacgctaatcttgaaccaaaagctgagcaatatgcggtcattacctggcgtgccctcaataagctgccactagttcctcaagtggcaaatgtagttgtgccaaccgctgtttatttctctgaaaagtacaacgatgttgttcgtggcactactgagcagggatatagagtgtcctcttatttgcctttgttgcccactgagaaaattactaaggtgtttggagatgaggcatcataa

SEQ ID NO: 19 - protein, 204 aa, Hevea brasiliensis
MAEEVEEERLKYLDFVRAAGVYAVDSFSTLYLYAKDISGPLKPGVDTIENVVKTVVTPVYYIPLEAVKFVDKTVDVSVTSLDGVVPPVIKQVSAQTYSVAQDAPRIVLDVASSVFNTGVQEGAKALYANLEPKAEQYAVITWRALNKLPLVPQVANVVVPTAVYFSEKYNDVVRGTTEQGYRVSSYLPLLPTEKITKVFGDEAS

SEQ ID NO: 20 - DNA, 20 bp, Artificial Sequence
  PRIMER 1
ctcaaggctaccttattggg

SEQ ID NO: 21 - DNA, 20 bp, Artificial Sequence
  PRIMER 2
ctcagcaattgcaacacctg

SEQ ID NO: 22 - DNA, 27 bp, Artificial Sequence
  PRIMER 3
ggtaccgctaccttattgggaactacc

SEQ ID NO: 23 - DNA, 26 bp, Artificial Sequence
  PRIMER 4
agatctaactcttcccatttcttccc

SEQ ID NO: 24 - DNA, 21 bp, Artificial Sequence
  PRIMER 5
gaatccatggcggatctgaag

SEQ ID NO: 25 - DNA, 20 bp, Artificial Sequence
  PRIMER 6
gtccatgtatctggataccc

SEQ ID NO: 26 - DNA, 24 bp, Artificial Sequence
  PRIMER 7
caagatgagttcagtgaatttggg

SEQ ID NO: 27 - DNA, 22 bp, Artificial Sequence
  PRIMER 8
tgcattagttttgcctgtgagc

SEQ ID NO: 28 - DNA, 21 bp, Artificial Sequence
  PRIMER 9
atttttacatggacaccaccg

SEQ ID NO: 29 - DNA, 21 bp, Artificial Sequence
  PRIMER 10
accagattcccactaagatgc

SEQ ID NO: 30 - DNA, 21 bp, Artificial Sequence
  PRIMER 11
tccatatatggacgaggttcg

SEQ ID NO: 31 - DNA, 20 bp, Artificial Sequence
  PRIMER 12
gcagctgtgttacccttcag

SEQ ID NO: 32 - DNA, 23 bp, Artificial Sequence
  PRIMER 13
cagtcgctccaaaatggatgtgc

SEQ ID NO: 33 - DNA, 24 bp, Artificial Sequence
  PRIMER 14
gattttcttaggaagaaggcttgg

SEQ ID NO: 34 - DNA, 23 bp, Artificial Sequence
  PRIMER 15
ctagctggtctataatggatgcc

SEQ ID NO: 35 - DNA, 25 bp, Artificial Sequence
  PRIMER 16
gaatcaatttacctcaatagaaggc

SEQ ID NO: 36 - DNA, 21 bp, Artificial Sequence
  PRIMER 17
ttccaccatgggtgaggctcc

SEQ ID NO: 37 - DNA, 22 bp, Artificial Sequence
  PRIMER 18
tctcaactcaacttgtgaatcg

SEQ ID NO: 38 - DNA, 24 bp, Artificial Sequence
  PRIMER 19
atggaattatacaacggtgagagg

SEQ ID NO: 39 - DNA, 25 bp, Artificial Sequence
  PRIMER 20
ttttaagtattccttatgtttctcc

SEQ ID NO: 40 - DNA, 20 bp, Artificial Sequence
  PRIMER 21
tatggctgaagaggtggagg

SEQ ID NO: 41 - DNA, 22 bp, Artificial Sequence
  PRIMER 22
tgatgcctcatctccaaacacc

SEQ ID NO: 42 - DNA, 30 bp, Artificial Sequence
  PRIMER 23
ctcgagaacaatggcggatctgaagtcaac

SEQ ID NO: 43 - DNA, 32 bp, Artificial Sequence
  PRIMER 24
ggtacctgtttctgtctcttgtaaattttggc

SEQ ID NO: 44 - DNA, 30 bp, Artificial Sequence
  PRIMER 25
ctcgagaacaatgagttcagtgaatttggg

SEQ ID NO: 45 - DNA, 35 bp, Artificial Sequence
  PRIMER 26
ggatccttgttttgcctgtgagcgatgtaattagc

SEQ ID NO: 46 - DNA, 32 bp, Artificial Sequence
  PRIMER 27
ctcgagacaaatggacaccaccggccggctcc

SEQ ID NO: 47 - DNA, 34 bp, Artificial Sequence
  PRIMER 28
ggtaccacagatgcagctttagacatatctttgc

SEQ ID NO: 48 - DNA, 33 bp, Artificial Sequence
  PRIMER 29
ctcgagacaaatggacgaggttcgccggcgacc

SEQ ID NO: 49 - DNA, 35 bp, Artificial Sequence
  PRIMER 30
ggtaccacgaaagttattttggatacatcttttgc

SEQ ID NO: 50 - DNA, 30 bp, Artificial Sequence
  PRIMER 31
aagcttacaaatggatgtgcgccggcgacc

SEQ ID NO: 51 - DNA, 29 bp, Artificial Sequence
  PRIMER 32
ggtaccacggaagaaggcttggaaacagc

SEQ ID NO: 52 - DNA, 30 bp, Artificial Sequence
  PRIMER 33
aagcttacaaatggatgcccgccggcgacc

SEQ ID NO: 53 - DNA, 33 bp, Artificial Sequence
  PRIMER 34
ggtaccacatttacctcaatagaaggcattgtc

SEQ ID NO: 54 - DNA, 32 bp, Artificial Sequence
  PRIMER 35
ctcgagaacaatgggtgaggctccagatgtcg

SEQ ID NO: 55 - DNA, 35 bp, Artificial Sequence
  PRIMER 36
ggtacctgactcaacttgtgaatcgttttcatgtc

SEQ ID NO: 56 - DNA, 28 bp, Artificial Sequence
  PRIMER 37
ctcgagccaacaatggaattatacaacg

SEQ ID NO: 57 - DNA, 33 bp, Artificial Sequence
  PRIMER 38
ggatcctcttttaagtattccttatgtttctcc

SEQ ID NO: 58 - DNA, 29 bp, Artificial Sequence
  PRIMER 39
ctcgagaacaatggctgaagaggtggagg

SEQ ID NO: 59 - DNA, 30 bp, Artificial Sequence
  PRIMER 40
ggatcctgtgatgcctcatctccaaacacc

SEQ ID NO: 60 - DNA, 417 bp, Hevea brasiliensis
atggctgaagacgaagacaaccaacaagggcagggggaggggttaaaatatttgggttttgtgcaagacgcggcaacttatgctgtgactaccttctcaaacgtctatctttttgccaaagacaaatctggtccactgcagcctggtgtcgatatcattgagggtccggtgaagaacgtggctgtacctctctataataggttcagttatattcccaatggagctctcaagtttgtagacagcacggttgttgcatctgtcactattatagatcgctctcttcccccaattgtcaaggacgcatctatccaagttgtttcagcaattcgagctgccccagaagctgctcgttctctggcttcttctttgcctgggcagaccaagatacttgctaaggtgttttatggagagaattga

SEQ ID NO: 61 - protein, 138 aa, Hevea brasiliensis
MAEDEDNQQGQGEGLKYLGFVQDAATYAVTTFSNVYLFAKDKSGPLQPGVDIIEGPVKNVAVPLYNRFSYIPNGALKFVDSTVVASVTIIDRSLPPIVKDASIQVVSAIRAAPEAARSLASSLPGQTKILAKVFYGEN

SEQ ID NO: 62 - DNA, 22 bp, Artificial Sequence
  PRIMER 41
atggctgaagacgaagacaacc

SEQ ID NO: 63 - DNA, 24 bp, Artificial Sequence
  PRIMER 42
attctctccataaaacaccttagc

SEQ ID NO: 64 - DNA, 32 bp, Artificial Sequence
  PRIMER 43
ctcgagaacaatggctgaagacgaagacaacc

SEQ ID NO: 65 - DNA, 32 bp, Artificial Sequence
  PRIMER 44
ggatccaaattctctccataaaacaccttagc

SEQ ID NO: 66 - DNA, 855 bp, Hevea brasiliensis
atggaattatacaacggtgagaggccaagtgtgttcagacttttagggaagtatatgagaaaagggttatatagcatcctaacccagggtcccatccctactcatattgccttcatattggatggaaacgggaggtttgctaagaagcataaactgccagaaggaggtggtcataaggctggatttttagctcttctgaacgtactaacttattgctatgagttaggagtgaaatatgcgactatctatgcctttagcatcgataattttcgaaggaaacctcatgaggttcagtacgtaatgaatctaatgctggagaagattgaagggatgatcatggaagaaagtatcatcaatgcatatgatatttgcgtgcgttttgttggtaatctgaagcttttagatgagccactcaagaccgcagcagataagataatgagggctactgccaaaaattccaaatttgtgcttctccttgctgtatgctacacttcaactgatgagatcgtgcatgctgttgaagaatcctctaaggataaattgaaatccgatgaaatttgcaacgatggaaacggagattgtgtgattaaaattgaggagatggagccatattctgaaataaaacttgtagagcttgagagaaacacttacataaatccttatcctgatgtcttgattcgaacttctggggagacccgtctgagcaactacctactttggcagactactaattgcatactgtattctcctcatgcactgtggccagagattggtcttcgacacgtggtgtgggcagtaattaactgccaacgtcattattcttacttggagaaacataaggaatacttaaaataa

SEQ ID NO: 67 - protein, 284 aa, Hevea brasiliensis
MELYNGERPSVFRLLGKYMRKGLYSILTQGPIPTHIAFILDGNGRFAKKHKLPEGGGHKAGFLALLNVLTYCYELGVKYATIYAFSIDNFRRKPHEVQYVMNLMLEKIEGMIMEESIINAYDICVRFVGNLKLLDEPLKTAADKIMRATAKNSKFVLLLAVCYTSTDEIVHAVEESSKDKLKSDEICNDGNGDCVIKIEEMEPYSEIKLVELERNTYINPYPDVLIRTSGETRLSNYLLWQTTNCILYSPHALWPEIGLRHVVWAVINCQRHYSYLEKHKEYLK

SEQ ID NO: 68 - DNA, 24 bp, Artificial Sequence
  PRIMER 45
atggaattatacaacggtgagagg

SEQ ID NO: 69 - DNA, 25 bp, Artificial Sequence
  PRIMER 46
ttttaagtattccttatgtttctcc

SEQ ID NO: 70 - DNA, 28 bp, Artificial Sequence
  PRIMER 47
ctcgagccaacaatggaattatacaacg

SEQ ID NO: 71 - DNA, 33 bp, Artificial Sequence; identical to SEQ ID NO: 57
  PRIMER 48